(19) United States
(12) Patent Application Publication
(10) Pub. No.: US 2008/0248122 A1
(43) Pub. Date: Oct. 9, 2008

(54) Microencapsules Containing Surface-Modified Microparticles And Methods Of Forming And Using The Same

(75) Inventors: Julia Rashba-Step, Newton, MA (US); Terrence L. Scott, Winchester, MA (US); Ramin Darvari, Lexington, MA (US)

Correspondence Address: BAXTER HEALTHCARE CORPORATION, ONE BAXTER PARKWAY, DF2-2E, DEERFIELD, IL, 60015, US

(73) Assignees: BAXTER INTERNATIONAL INC., Deerfield, IL (US); BAXTER HEALTHCARE S.A., Wallisellen (CH)

(21) Appl. No.: 11/869,715

(22) Filed: Oct. 9, 2007

Related U.S. Application Data
(60) Provisional application No. 60/828,507, filed on Oct. 6, 2006.
(60) Provisional application No. 60/863,901, filed on Nov. 1, 2006.

(51) Int. Cl. A61K 9/16; A61K 38/28; A61K 38/18

(52) U.S. Cl. 424/490; 514/3; 514/12

(57) ABSTRACT

An encapsulated particle, comprising a plurality of preformed microparticles encapsulated in an amorphous matrix, wherein at least one of the preformed microparticles comprises a core microparticle and a monolayer associated with the outer surface of the core microparticle. The core microparticle comprises at least one active agent that is capable of being released from the encapsulated particle.

### CROSS-REFERENCE TO RELATED APPLICATION

The benefits under 35 U.S.C. §119(e) of U.S. provisional patent application Ser. Nos. 60/828,507 filed Oct. 6, 2006, and 60/863,901 filed Nov. 1, 2006, the entire disclosures of which are incorporated herein by reference, are hereby claimed.

### FIELD AND BACKGROUND

The present disclosure is generally directed to microparticles containing one or more active agents and delivery methods of such microparticles to subjects. More particularly, the present disclosure is directed to surface-modified microparticles that are further encapsulated in matrices to form microencapsules. The present disclosure is further directed to methods for making and using such microencapsules.

Microparticles have been used in many different applications, including the controlled delivery and/or release of active agents. Controlling or modifying the release profile of an active agent can, if desired, prolong the levels (such as therapeutic levels) of the active agent in the blood stream of the recipient, improve pharmacokinetics and pharmacodynamics, and result in greater convenience to the recipient.

### SUMMARY

The present disclosure is generally directed to methods for preparing particles such as microencapsules. In one example, the method includes providing preformed microparticles that include at least one active agent. The preformed microparticle has an outer surface carrying a net surface charge. The method further includes exposing at least the outer surface of the preformed microparticle to at least one charged compound having a net charge that is opposite in sign to the net surface charge of the preformed microparticle outer surface. A monolayer of the charged compound is formed whereby the monolayer is associated with the preformed microparticle outer surface. The method further includes encapsulating one or more of the surface-modified microparticles to form microencapsules.

The present disclosure is also directed to a method for preparing microencapsules by forming a microparticle containing the active agent, forming a monolayer including at least one charged compound on the formed microparticle, thereby preparing a surface-modified microparticle, and encapsulating one or more of the surface-modified microparticles, optionally in combination with one or more of the solid and amorphous microparticles, to form microencapsules. The microencapsules have in vitro and/or in vivo release profiles that are different from those of the surface-modified microparticles encapsulated therein, and are further different from those of the core microparticles therein.

The present disclosure further describes microencapsules that display a synergistic reduction in initial burst in vitro and/or in vivo release of the active agent therein that is unexpected from the combined effects of surface modification and microencapsulation. The microencapsules further display extended and/or sustained release profiles that are not adversely affected by the combination of surface modification and microencapsulation.

The present disclosure is also directed to a method for preparing microencapsules that includes providing preformed microparticles including at least one active agent, the preformed microparticles have an outer surface carrying a net surface charge, further exposing at least the outer surface of the preformed microparticles to at least one charged compound having a net charge that is opposite in sign to the net surface charge of the preformed microparticle. An intermediate microparticle is formed that includes the preformed microparticle and a formed monolayer including the at least one charged compound wherein the formed monolayer is associated with the preformed microparticle outer surface. The formed monolayer is then exposed to at least a different charged compound to form a surface modified microparticle that includes the intermediate microparticle and a subsequent monolayer including at least the one different charged compound, whereby the surface modified microparticle has a release profile of the at least one active agent that is different from the release profile of the intermediate microparticle. One or more of the surface modified microparticles and/or one or more of the preformed microparticles are then encapsulated to form microencapsules. The microencapsule has in vitro and/or in vivo release profiles that are different from those of the surface-modified microparticle.

The present disclosure is also directed to a microencapsule that includes a plurality of preformed microparticles encapsulated in an amorphous matrix. The preformed microparticle includes a core microparticle and a monolayer associated with the outer surface of the core microparticle. The core microparticle includes at least one active agent that is capable of being released from the microencapsule.

The present disclosure is also directed to a microencapsule that includes a plurality of preformed microparticles encapsulated in an amorphous matrix. The preformed microparticle includes a core microparticle and, optionally, one or more monolayers separating the core microparticle from the amorphous matrix. The core microparticle includes at least one active agent that is capable of being released from the microencapsule.

The present disclosure is also directed to a microencapsule that includes a core microparticle that is 80% or greater by weight of at least one active agent. The outer surface of the core microparticle carries a selected net surface charge. A monolayer of at least one charged compound carrying a net surface charge that is sufficiently different from the surface charge of the core microparticle outer surface to allow for association therewith, is associated with the core microparticle outer surface at least by, but not limited to, electrostatic interaction with the outer surface. One or more of the surface-modified microparticles and/or the core microparticles are encapsulated within a matrix of one or more polymers.

The present disclosure is also directed to a microencapsule including at least 5% by weight, such as 10%, 20%, 50%, or more by weight, of at least one active agent and displaying a 1-hour percentage of cumulative release of the active agent that is 10% or less, such as 5% or less, when subjected to in vitro release in a suitable buffer at selected pH and temperature. The microencapsule can display in the same in vitro release buffer a 24-hr percentage of cumulative release of the active agent that is 10% or less, such as 5% or less. The microencapsule can have an in vivo release profile that is uncorrelated to its in vitro release profile. The microencapsule is suitable for in vivo administration. Upon such administration, the microencapsule provides a Cₘₐₓ and tₘₐₓ that is different from the Cₘₐₓ and tₘₐₓ of the surface-modified microparticles encapsulated therein, and is different from the Cₘₐₓ and tₘₐₓ of the core microparticle within the surface-modified microparticle.

Further details of the above-described microencapsules, methods of making microencapsules and methods for controlling the release of active agents from the microencapsules are discussed below.

### BRIEF DESCRIPTION OF THE FIGURES

FIG. 1 is a graph showing in vitro release profiles of microparticles and microencapsules of different formulations (Example 1);

FIG. 2 is a graph showing in vivo release profiles of microparticles and microencapsules of different formulations (Example 1) wherein the graph on the right is the portion of the graph on the left showing the release over approximately 25 hours;

FIG. 3 is a graph showing in vitro release profiles of microparticles and microencapsules of different formulations (Example 2);

FIG. 4 is a graph showing in vivo release profiles of microparticles and microencapsules of different formulations (Example 2) wherein the graph on the right is a portion of the graph on the left showing the release over approximately 25 hours;

FIG. 5 is a graph showing the in vitro release profiles of microencapsules including mixtures of coated and uncoated microparticles (Example 3); and

FIG. 6 is a schematic representation of an exemplary method set forth in the present disclosure.

### DETAILED DESCRIPTION

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Thus, for example, the reference to a particular microparticle is a reference to one such microparticle or a plurality of such microparticles, including equivalents thereof known to one skilled in the art. Also, as used herein and in the claims, the terms “at least one” and “one or more” have the same meaning and include one, two, three or more. The following terms, unless otherwise indicated, shall be understood to have the following meanings when used in the context of the present disclosure.

“Active agent” refers to naturally occurring, synthetic, or semi-synthetic materials (e.g., compounds, fermentates, extracts, cellular structures) capable of eliciting, directly or indirectly, one or more physical, chemical, and/or biological effects, in vitro and/or in vivo. The active agent may be capable of preventing, alleviating, treating, and/or curing abnormal and/or pathological conditions of a living body, such as by destroying a parasitic organism, or by limiting the effect of a disease or abnormality by materially altering the physiology of the host or parasite. The active agent may be capable of maintaining, increasing, decreasing, limiting, or destroying a physiological body function. The active agent may be capable of diagnosing a physiological condition or state by an in vitro and/or in vivo test. The active agent may be capable of controlling or protecting an environment or living body by attracting, disabling, inhibiting, killing, modifying, repelling and/or retarding an animal or microorganism. The active agent may be capable of otherwise treating (such as deodorizing, protecting, adorning, grooming) a body. Depending on the effect and/or its application, the active agent may further be referred to as a bioactive agent, a pharmaceutical agent (such as a prophylactic agent, a therapeutic agent), a diagnostic agent, a nutritional supplement, and/or a cosmetic agent, and includes, without limitation, prodrugs, affinity molecules, synthetic organic molecules, polymers, low molecular weight molecules (such as those having a molecular weight of 2 kD or less, for example, 1.5 kD or less, or 1 kD or less), macromolecules (such as those having a molecular weight of 2 kD or greater, preferably 5 kD or greater), proteinaceous compounds, peptides, vitamins, steroids, steroid analogs, lipids, nucleic acids, carbohydrates, precursors thereof, and derivatives thereof. Active agents may be ionic or non-ionic, may be neutral, positively charged, negatively charged, or zwitterionic, and may be used singly or in combination of two or more thereof. Active agents may be water insoluble, but more preferably are water-soluble. Active agents may have an isoelectric point of 7.0 or greater, but preferably have an isoelectric point of less than 7.0.

“Microparticle” refers to a particulate that is solid (including substantially solid or semi-solid, but excluding gel, liquid and gas), having an average geometric particle size (sometimes referred to as diameter) of less than 1 mm, preferably 200 microns or less, more preferably 100 microns or less, most preferably 10 microns or less. In one example, the particle size may be 0.01 microns or greater, preferably 0.1 microns or greater, more preferably 0.5 microns or greater, and most preferably from 0.5 microns to 5 microns. Average geometric particle size may be measured by dynamic light scattering methods (such as photocorrelation spectroscopy, laser diffraction, low-angle laser light scattering (LALLS), medium-angle laser light scattering (MALLS), light obscuration methods (such as Coulter analysis method), or other methods (such as rheology, light or electron microscopy). Particles for pulmonary delivery will have an aerodynamic particle size determined by time of flight measurements or Andersen Cascade Impactor measurements. Microparticles may have a spherical shape (sometimes referred to as microspheres) and/or may be encapsulated (sometimes referred to as microencapsules). Certain microparticles may have one or more internal voids and/or cavities. Other microparticles may be free of such voids or cavities. Microparticles may be porous or, preferably non-porous. Microparticles may be formed from, in part or in whole, one or more non-limiting materials, such as the active agents, carriers, polymers, stabilizing agents, and/or complexing agents disclosed herein.

“Proteinaceous compounds” refer to natural, synthetic, semi-synthetic, or recombinant compounds of or related structurally and/or functionally to proteins, such as those containing or consisting essentially of α-amino acids covalently associated through peptide linkages. Non-limiting proteinaceous compounds include globular proteins (e.g., albumins, globulins, histones), fibrous proteins (e.g., collagens, elastins, keratins), compound proteins (including those containing one or more non-peptide components, e.g., glycoproteins, nucleoproteins, mucoproteins, lipoproteins, metalloproteins), therapeutic proteins, fusion proteins, receptors, antigens (such as synthetic or recombinant antigens), viral surface proteins, hormones and hormone analogs, antibodies (such as monoclonal or polyclonal antibodies), enzymes, Fab fragments, cyclic peptides, linear peptides, and the like. Non-limiting therapeutic proteins include bone morphogenic proteins, drug resistance proteins, toxoids, erythropoietins, proteins of the blood clotting cascade (e.g., Factor VII, Factor VIII, Factor IX, et al.), subtilisin, ovalbumin, alpha-1-antitrypsin (AAT), DNase, superoxide dismutase (SOD), lysozymes, ribonucleases, hyaluronidase, collagenase, human growth hormone (hGH), erythropoietin, insulin, insulin-like growth factors, interferons, glatiramer, granulocyte-macrophage colony-stimulating factor, granulocyte colony-stimulating factor, desmopressin, leutinizing hormone release hormone (LHRH) agonists (e.g., leuprolide, goserelin, buserelin, gonadorelin, histrelin, nafarelin, deslorelin, fertirelin, triptorelin), LHRH antagonists, vasopressin, cyclosporine, calcitonin, parathyroid hormone, parathyroid hormone peptides, glucogen-like peptides, and analogs thereof. Proteinaceous compounds may be neutral, positively charged, negatively charged, or zwitterionic, and may be used singly or in combination of two or more thereof.

“Nucleic acids” refer to natural, synthetic, semi-synthetic, or recombinant compounds formed at least in part from two or more of the same or different nucleotides, and may be single-stranded or double-stranded. Non-limiting examples of nucleic acids include oligonucleotides (such as those having 20 or less base pairs, e.g., sense, anti-sense, or missense), aptamers, polynucleotides (e.g., sense, anti-sense, or missense), DNA (e.g., sense, anti-sense, or missense), RNA (e.g., sense, anti-sense, or missense), siRNA, nucleotide acid constructs, single-stranded or double-stranded segments thereof, as well as precursors and derivatives thereof (e.g., glycosylated, hyperglycosylated, PEGylated, FITC-labeled, nucleosides, salts thereof). Nucleic acids may be neutral, positively charged, negatively charged, or zwitterionic, and may be used singly or in combination of two or more thereof.

“Macromolecule” refers to a material capable of providing a three-dimensional (e.g., tertiary and/or quaternary) structure, and includes carriers and certain active agents of the present disclosure. Non-limiting macromolecules used to form the microparticles include, inter alia, polymers, copolymers, proteinaceous compounds (as defined herein, for example, including proteins such as enzymes, recombinant proteins, albumins like human serum albumin, as well as peptides), lipids, carbohydrates, polysaccharides, nucleic acids, vectors (e.g., virus, viral particles), complexes and conjugates thereof (e.g., by covalent and/or non-covalent associations, between two macromolecules like carbohydrate-protein conjugates, between an active agent and a macromolecule like hapten-protein conjugates, the active agent may or may not be capable of providing a tertiary and/or quaternary structure), and mixtures of two or more thereof, preferably having a molecular weight of 1,500 or greater. Macromolecules may be neutral, positively charged, negatively charged, or zwitterionic, and may be used singly or in combination of two or more thereof.

“Spherical” refers to a geometric shape that is at least “substantially spherical.” “Substantially spherical” means that the ratio of the longest length (i.e., the distance between two points on the perimeter capable of being connected through the geometric center of the shape) to the shortest length on any cross-section that passes through the geometric center is about 1.5 or less, preferably about 1.33 or less, more preferably 1.25 or less. Spherical does not require a line of symmetry. Further, the microparticles may have surface texturing (such as continuous or discrete lines, islands, lattice, indentations, channel openings, protuberances that are small in scale when compared to the overall size of the microparticles) and still be spherical. Surface contact there between is minimized in microparticles that are spherical, which minimizes the undesirable agglomeration of the microparticles. In comparison, microparticles that are crystals or flakes typically display significant agglomeration through ionic and/or non-ionic interactions at relatively large flat surfaces.

“Monodisperse size distribution” refers to a preferred microparticle size distribution in which the ratio of the volume diameter of the 90^{th} percentile (i.e., the average particle size of the largest 10% of the microparticles) to the volume diameter of the 10^{th} percentile (i.e., the average particle size of the smallest 10% of the microparticles) is about 5 or less, preferably about 3 or less, more preferably about 2 or less, most preferably about 1.5 to 1. Consequently, “polydisperse size distribution” refers to one where the diameter ratio described above is greater than 5, preferably greater than 8, more preferably greater than 10. In microparticles having a polydisperse size distribution, smaller microparticles may fill in the gaps between larger microparticles, thus possibly displaying large contact surfaces and significant agglomeration there between. A Geometric Standard Deviation (GSD) of 2.5 or less, preferably 1.8 or less, may also be used to indicate a monodisperse size distribution. Calculation of GSD is known and understood to one skilled in the art.

“Amorphous” refers to materials and constructions that are “substantially amorphous,” such as microparticles having multiple non-crystalline domains (or lacking crystallinity altogether) or otherwise non-crystalline. Substantially amorphous microparticles of the present disclosure are generally random solid particulates in which crystalline lattices constitute less than 50% by volume and/or weight of the microparticles, or are absent, and include semi-crystalline microparticles and non-crystalline microparticles as understood by one skilled in the art.

“Solid” refers to a state that includes at least substantially solid and/or semi-solid, but excludes gel, liquid, and gas.

“Preformed microparticle” refers to a microparticle fabricated using one or more non-limiting methods, such as those known to one skilled in the art, without surface modification as described herein, having or capable of having on its outer surface a net surface electric charge that is positive, negative, or neutral. A preformed microparticle is also referred to herein as a “core microparticle” or a “core.” The preformed or core microparticle typically comprises one or more active agents and, optionally, one or more carriers, which, independently, may be compartmentalized in a portion of the preformed or core microparticle or preferably be distributed substantially homogeneously throughout the preformed microparticles. The net surface charge, preferably being non-zero, may be contributed primarily or at least substantially by the active agent(s) and/or the optional carrier(s) present in the preformed microparticles. The preformed microparticles may be amorphous and solid.

“Carrier” refers to a compound, typically a macromolecule, having a primary function to provide a three-dimensional structure (including tertiary and/or quaternary structure). The carrier may be unassociated or associated with the active agent (such as conjugates or complexes thereof) in forming microparticles as described above. The carrier may further provide other functions, such as being an active agent, modifying a release profile of the active agent from the microparticle, and/or imparting one or more particular properties to the microparticle (such as contribute at least in part to the net surface charge). In one example, the carrier is a protein (such as albumins, preferably human serum albumin) having a molecular weight of 1500 Daltons or greater.

“Polymer” or “polymeric” refers to a natural, synthetic, or semi-synthetic molecule having two or more repeating monomer units in a main chain or ring structure. Polymers broadly include dimers, trimers, tetramers, oligomers, higher molecular weight polymers, adducts, homopolymers, random copolymers, pseudo-copolymers, statistical copolymers, alternating copolymers, periodic copolymers, bipolymers, terpolymers, quaterpolymers, other forms of copolymers, substituted derivatives thereof, and mixtures thereof, and narrowly refer to molecules having 10 or more repeating monomer units. Polymers may be linear, branched, block, graft, monodisperse, polydisperse, regular, irregular, tactic, isotactic, syndiotactic, stereoregular, atactic, stereoblock, single-strand, double-strand, star, comb, dendritic, and/or ionomeric, may be ionic or non-ionic, may be neutral, positively charged, negatively charged, or zwitterionic, and may be used singly or in combination of two or more thereof.

“Charged” and “electrically charged” refer interchangeably to the capability of providing one, two, three, or more formal units of electrical charges of the same or opposite sign and/or the presence of such charges (i.e., “charged” refers to chargeable and/or charged). The electrical charges may be provided by and/or be present in the form of one or more of the same or different organic and/or organometallic moieties (e.g., ionic groups, ionizable groups, precursors thereof) in a compound (e.g., polyelectrolytes, proteins) or the structure (e.g., the preformed microparticle, the monolayers) of interest, preferably when subjected to certain conditions (such as in solution or suspension).

“Charged compound” and “electrically charged compound” refer interchangeably to a single compound that is charged as described above, or a combination of two or more different compounds in unassociated and/or associated forms (e.g., conjugates, aggregates, and/or complexes thereof), each of which independently has and/or is capable of having a net charge of the same sign.

“Monolayer” refers to a single layer formed over a three-dimensional substrate, from a composition of one or more compounds (such as a charged compound as described above). The monolayer may be a continuous and nonporous monolayer, a continuous and porous monolayer (such as a lattice network), a non-continuous monolayer of a plurality of discrete elements (e.g., islands, strips, clusters, etc.), or a combination thereof. Typically, the monolayer will be decomposable or degradable, such as biodegradable, enzymatically or hydrolytically degradable and the like, to allow for non-diffusional release of an active agent (from the microparticle) over which the monolayer is deposited. The monolayer may have a thickness of 100 nm or less, preferably 50 nm or less, more preferably 20 nm or less, most preferably 10 nm or less. In one example, the monolayer is formed through self-assembly of a charged compound.

“Saturated monolayer” refers to a monolayer as defined above that is incapable of further incorporating, cumulatively, an additional amount of the composition forming the monolayer when subjected to the same set of conditions under which the monolayer is formed. Saturated monolayers are preferred monolayers for use in surface-modified microparticles.

“Net charge” and “net electric charge” are used interchangeably and refer to the sum of all formal units of electric charge a charged compound is capable of having or has, such as in a flowable medium under certain conditions (preferably in a solution of certain pH). The net charge may be positive, negative, or zero (such as in zwitterionic compounds), and is condition-dependent (e.g., solvent, pH).

“Net surface charge” and “net surface electric charge” are used interchangeably and refer to an overall cumulative electric charge on an outermost surface of a three-dimensional structure (e.g., a microparticle, a monolayer). The net surface charge may be positive, negative, or zero, and is condition-dependent (e.g., solvent, pH).

“Ambient temperature” refers to a temperature around room temperature, typically in a range of about 20° C. to about 40° C.

“Therapeutic” refers to any pharmaceutic, drug, prophylactic agent, contrast agent, or dye useful in the treatment (including prevention, diagnosis, alleviation, suppression, remission, or cure) of a malady, affliction, disease or injury in a subject. Therapeutically useful peptides and nucleic acids may be included within the meaning of the term “pharmaceutic” or “drug.”

“Diagnostic agent” refers to any material or substance useful in connection with methods for perceptually observing (e.g., imaging) a normal or abnormal biological condition or state, or detecting the presence or absence of a pathogen or a pathological condition. Non-limiting diagnostic agents include contrast agents and dyes for use in connection with radiography imaging (e.g., X-ray imaging), ultrasound imaging, magnetic resonance imaging, computed tomography, positron emission tomography imaging, and the like. Diagnostic agents further include any other agents useful in facilitating diagnosis in vivo and/or in vitro, whether or not imaging methodology is employed.

“Cross-link,” “cross-linked” and “cross-linking” generally refer to the linking of two or more materials and/or substances, including any of those disclosed herein, through one or more covalent and/or non-covalent (e.g., ionic) associations. Cross-linking may be effected naturally (e.g., disulfide bonds of cystine residues) or through synthetic or semi-synthetic routes, for example, optionally in the presence of one or more cross-linkers (i.e., a molecule X by itself capable of reacting with two or more materials/substances Y and Z to form a cross-link product Y-X-Z, where the associations of Y-X and X-Z are independently covalent and/or non-covalent), initiators (i.e., a molecule by itself capable of providing reactive species like free radicals for the cross-link reaction, e.g., thermally decomposable initiators like organic peroxides, azo initiators, and carbon-carbon initiators, actinically decomposable initiators like photoinitiators of various wavelengths), activators (i.e., a molecule A capable of reacting with a first material/substance Y to form an activated intermediate [A-Y], which in turn reacts with a second material/substance Z to form a cross-link product Y-Z, while A is chemically altered or consumed during the process), catalysts (i.e., a molecule capable of modifying the kinetics of the cross-link reaction without being chemically modified during the process), co-agents (i.e., a molecule that, when co-present with one or more of the initiators, activators, and/or catalysts, is capable of modifying the kinetics of the cross-link reaction and/or being incorporated into the cross-link product of the two or more materials/substances, but otherwise is non-reactive to the materials/substances), and/or energy sources (e.g., heating; cooling; high-energy radiations like electromagnetic, e-beam, and nuclear; acoustic radiations like ultrasonic; etc.).

“Covalent association” refers to an intermolecular interaction (e.g., a bond) between two or more individual molecules that involves the sharing of electrons in the bonding orbitals of two atoms.

“Non-covalent association” refers to an intermolecular interaction between two or more individual molecules which does not involve a covalent bond. Intermolecular interaction depends on, for example, polarity, electric charge, and/or other characteristics of the individual molecules, and includes, without limitation, electrostatic (e.g., ionic) interactions, dipole-dipole interactions, van der Waal's forces, and combinations of two or more thereof.

“Electrostatic interaction” refers to an intermolecular interaction between two or more positively or negatively charged moieties/groups, which may be attractive when two are oppositely charged (i.e., one positive, another negative), repulsive when two charges are of the same sign (i.e., two positive or two negative), or a combination thereof.

“In association with” and “associated with” refer in general to the one or more interactions between, and/or incorporation of, different materials (typically those that are part of the microparticles), one or more of such materials and one or more structures (or portions thereof) of the microparticles, and different structures (or portions thereof) of the microparticles. The materials of the microparticles include, without limitation, ions such as monovalent and polyvalent ions disclosed herein, as well as compounds such as active agents, stabilizing agents, cross-link agents, charged or uncharged compounds, the various polymers disclosed herein, and combinations of two or more thereof. The structures of the microparticles and portions thereof include, without limitation, core, core microparticle, preformed microparticle, monolayer, intermediate microparticle, surface-modified microparticle, portions of such structures (such as outer surfaces, inner surfaces), domains between such structures and portions thereof, and combinations of two or more thereof. Various associations, being reversible or irreversible, migratory or non-migratory, may be present singly or in combination of two or more thereof. Non-limiting associations include, without limitation, covalent and/or non-covalent associations (e.g., covalent bonding, ionic interactions, electrostatic interactions, dipole-dipole interactions, hydrogen bonding, van der Waal's forces, cross-linking, and/or any other interactions), encapsulation in layer/membrane, compartmentalization in the center or vesicles or between two layers/membranes, homogeneous integration throughout the microparticle or in a portion thereof (e.g., containment in, adhesion to, and/or affixation to the center or a layer or vesicle or an inner and/or outer surface thereof, interspersion, conjugations, and/or complexation between different materials).

“Controlled release” refers to a predetermined in vivo and/or in vitro release (e.g., dissolution) profile of an active agent, as compared to the release profile of the active agent in its native form. The active agent is preferably associated with a microparticle or a composition or formulation containing such a microparticle, as disclosed herein, such that one or more aspects of its release kinetics (e.g., initial burst, quantity and/or rate over a specified time period or phase, cumulative quantity over a specific time period, length of time for total release, pattern and/or profile, etc.) are increased, decreased, shortened, prolonged, and/or otherwise modified as desired. Non-limiting examples of controlled release include immediate/instant release (i.e., initial burst or rapid release), extended release, sustained release, prolonged release, delayed release, modified release, and/or targeted release, occurring individually, in combination of two or more thereof, or in the absence of one or more thereof (e.g. extended or sustained release in the absence of an initial burst).

“Extended release” refers to the release of an active agent, preferably in association with a microparticle or a composition or formulation containing such a microparticle, as disclosed herein, over a time period longer than the free aqueous diffusion period of the active agent in its native form. The extended release period may be hours (e.g., at least about 1, 2, 5, or 10 hours), days (e.g., at least about 1, 2, 3, 4, 5, 6, 7, 8, 10, 15, 20, 30, 40, 45, 60, or 90 days), weeks (at least about 1, 2, 3, 4, 5, 6, 10, 15, 20, 30, 40, or 50 weeks), months (at least about 1, 2, 3, 4, 6, 9, or 12 months), about 1 or more years, or a range between any two of the time periods. The pattern of an extended release may be continuous, periodic, sporadic, or a combination thereof.

“Sustained release” refers to an extended release of an active agent such that a functionally significant level of the active agent (i.e., a level capable of bring about the desired function of the active agent) is present at any time point of the extended release period, preferably with a continuous and/or uniform release pattern. Non-limiting examples of sustained release profiles include those, when displayed in a plot of release time (x-axis) versus cumulative release (y-axis), showing at least one upward segment that is linear, step-wise, zigzagging, curved, and/or wavy, over a time period of 1 hour or longer.

Other than in the operating examples, or unless otherwise expressly specified, all of the numerical ranges, amounts, values and percentages such as those for quantities of materials, times, temperatures, reaction conditions, ratios of amounts, values for molecular weight (whether number average molecular weight Mₙ or weight average molecular weight M_{w}), and others disclosed herein should be understood as modified in all instances by the term “about.” Accordingly, unless indicated to the contrary, the numerical parameters set forth in the present disclosure and attached claims are approximations that may vary. At the very least, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used.

“Formed from” and “formed of” denote open language. As such, it is intended that a composition “formed from” or “formed of” a list of recited components be a composition comprising at least these recited components, which can further include other non-recited components during formulation of the composition.

Examples provided herein, including those following “such as” and “e.g.,” are considered as illustrative only of various aspects of the present disclosure and embodiments thereof, without being specifically limited thereto. Any suitable equivalents, alternatives, and modifications thereof (including materials, substances, constructions, compositions, formulations, means, methods, conditions, etc.) known and/or available to one skilled in the art may be used or carried out in place of or in combination with those disclosed herein, and are considered to fall within the scope of the present disclosure.

In one example, each of the surface-modified microparticles of the present disclosure preferably contains an amorphous (e.g., such as free of crystalline structures) and solid preformed microparticle associated, at least at its outer surface, with at least one monolayer containing at least one charged compound. The preformed microparticle contains at least one active agent and/or at least one macromolecule having a molecular weight of 4,500 Daltons or greater. The macromolecule may be the active agent or may be different from the active agent. The macromolecule may be a carrier, a stabilizing agent, or a complexing agent (e.g., proteinaceous compounds, polyelectrolytes). The active agent and/or the macromolecule may constitute 40% to 100% or less, and typically at least 80%, such as 90% or more, or 95% or more, by weight of the preformed microparticle. Preferably, the active agent and/or the macromolecule is/are distributed homogeneously throughout the core microparticle. An outer surface of the preformed microparticle carries a net surface charge, which may be attributed, at least in part, and more typically in large part, to the active agent and/or the macromolecule, especially when the outer surface is formed of the active agent and/or the macromolecule. The preformed microparticle may be free of covalent crosslinking, hydrogels, lipids, and/or encapsulation. Alternatively, the preformed microparticle may contain one or more charged compounds, covalent crosslinking, and/or encapsulation. The one or more charged compounds in the preformed microparticle may be distributed homogeneously throughout the preformed microparticle, or compartmentalized in specific portions thereof, such as in a layer. The preformed microparticle may preferably have a particle size of 10 μm or less, and may have a monodisperse or polydisperse size distribution.

Methods of pre-forming the preformed microparticle are not particularly limited, and include those disclosed in U.S. Pat. No. 6,458,387 and U.S. Patent Publication No. 2005/0142206, which are incorporated herein by reference in their entirety. In one example, a single flowable continuous phase system (such as liquid, gas, or plasma, preferably a solution or suspension) is formulated to contain one or more active agents, a medium, and one or more phase-separation enhancing agents (PSEAs). The medium is preferably a liquid solvent (e.g., hydrophilic or hydrophobic organic solvents, water, buffers, aqueous-miscible organic solvents, and combinations of two or more thereof), more preferably an aqueous or aqueous-miscible solvent. The active agent and the PSEA may independently be dissolved, suspended, or otherwise homogeneously distributed within the medium. When subjecting the flowable system to certain conditions (such as a temperature below the phase transition temperature of the active agent in the medium), the active agent undergoes a liquid-solid phase separation and forms a discontinuous, preferably solid, phase (such as a plurality of core microparticles suspended in the medium), while the PSEA remains in the continuous phase (such as being dissolved in the medium).

The medium can be organic, containing an organic solvent or a mixture of two or more inter-miscible organic solvents, which may independently be aqueous-miscible or aqueous-immiscible. The solution can also be an aqueous-based solution containing an aqueous medium or an aqueous-miscible organic solvent or a mixture of aqueous-miscible organic solvents or combinations thereof. Suitable organic solvents include, without limitation, methylene chloride, chloroform, acetonitrile, ethylacetate, methanol, ethanol, alkanes such as pentane, hexane, heptane, octane, nonane, decane, the likes thereof, and combinations of two or more thereof (such as a 1:1 mixture of methylene chloride and acetone). The aqueous medium can be water, a buffer (e.g., normal saline, buffered solutions, buffered saline), and the like. Suitable aqueous-miscible organic solvents may be monomers or polymers, and include, but are not limited to, N-methyl-2-pyrrolidinone (N-methyl-2-pyrrolidone), 2-pyrrolidinone (2-pyrrolidone), 1,3-dimethyl-2-imidazolidinone (DMI), dimethylsulfoxide, dimethylacetamide, acetic acid, lactic acid, acetone, methyl ethyl ketone, acetonitrile, methanol, ethanol, n-propanol, isopropanol, 3-pentanol, benzyl alcohol, glycerol, tetrahydrofuran (THF), polyethylene glycol (PEG, e.g., PEG-4, PEG-8, PEG-9, PEG-12, PEG-14, PEG-16, PEG-120, PEG-75, PEG-150), PEG esters (e.g., PEG-4 dilaurate, PEG-20 dilaurate, PEG-6 isostearate, PEG-8 palmitostearate, PEG-150 palmitostearate), PEG sorbitans (such as PEG-20 sorbitan isostearate), PEG ethers (such as monoalkyl and dialkyl ethers, e.g., PEG-3 dimethyl ether, PEG-4 dimethyl ether, and glycofurol), polypropylene glycol (PPG), PPG esters (such as polypropylene glycol alginate (PGA), PPG dicaprylate, PPG dicaprate, PPG laurate), alkoxylated linear alkyl diols (such as PPG-10 butanediol), alkoxylated alkyl glucose ether (e.g., PPG-10 methyl glucose ether, PPG-20 methyl glucose ether), PPG alkyl ethers (such as PPG-15 stearyl ether), alkanes (e.g., propane, butane, pentane), and combinations of two or more thereof.

In a preferred example, a solution of the PSEA in a first solvent is provided, in which the PSEA is soluble in or miscible with the first solvent. The active agent is mixed with the first solution, either directly or by addition of a second solution in a second solvent. The first and second solvent may be the same or at least miscible with each other. Preferably the active agent is added at a temperature equal to or lower than ambient temperature, particularly when the active agent is a heat labile molecule such as certain proteinaceous compounds. However, the system may be heated to increase solubility of the active agent in the system, as long as the activity of the active agent is not adversely affected.

When the mixture is brought to phase separation conditions, the PSEA, while remaining in the liquid continuous phase, enhances and/or induces a liquid-solid phase separation of the active agent from the solution (such as by reducing solubility of the active agent), thereby forming the core microparticles (the solid discontinuous phase), which may preferably be microspheres. Suitable PSEA compounds include, but are not limited to, natural and synthetic polymers, linear polymers, branched polymers, cyclo-polymers, copolymers (random, block, grafted, such as poloxamers, particularly PLURONIC® F127 and F68), terpolymers, amphiphilic polymers, carbohydrate-based polymers, polyaliphatic alcohols, poly(vinyl)polymers, polyacrylic acids, polyorganic acids, polyamino acids, polyethers, polyesters, polyimides, polyaldehydes, polyvinylpyrrolidone (PVP), and surfactants. Suitable or exemplary PSEAs include, without limitation, polymers acceptable as pharmaceutical additives, such as PEGs (e.g., PEG 200, PEG 300, PEG 3350, PEG 8000, PEG 10000, PEG 20000, etc.), poloxamers, PVP, hydroxyethylstarch, amphiphilic polymers, as well as non-polymers (such as mixtures of propylene glycol and ethanol).

Conditions capable of enhancing, inducing, promoting, controlling, suppressing, retarding, or otherwise affecting the liquid-solid phase separation include, without limitation, changes in temperature, pressure, pH, ionic strength and/or osmolality of the solutions, concentrations of the active agent and/or the PSEA, the likes thereof, as well as rates of such changes, and combinations of two or more thereof. Such conditions may desirably be applied before and up to the phase separation, or even during the phase separation. In one example, the system is exposed to a temperature below the phase transition temperature of the active agent therein, alone or in combination with adjustments to the concentrations of the active agent and/or the PSEA, as described in U.S. Patent Publication No. 2005/0142206, the entire disclosure of which is incorporated herein by reference. The rate of temperature drop may be held constant or altered in any controlled manner, as long as it is within a range of 0.2° C./minute to 50° C./minute, preferably 0.2° C./minute to 30° C./minute. Freezing point depressing agents (FPDAs), used individually or in combination of two or more thereof, may be mixed in the system directly or in solutions (such as aqueous solutions) thereof, particularly for systems in which the freezing point is higher than the phase transition temperature of the active agent. Suitable FPDAs include, without limitation, propylene glycol, sucrose, ethylene glycol, alcohols (e.g., ethanol, methanol), and aqueous mixtures thereof.

In one example, the preformed microparticles may further comprise one or more excipients that negligibly affect the phase separation. The excipient may imbue the core microparticles and/or the compounds therein (e.g., the active agent, the optional carrier) with additional characteristics such as increased stability, controlled release of the active agent from the preformed microparticles, and/or modified permeation of the active agent through biological tissues. Suitable excipients include, but are not limited to, carbohydrates (e.g., trehalose, sucrose, mannitol), polyvalent cations (preferably metal cations, e.g., Zn²⁺, Mg²⁺, Ca²⁺, Cu²⁺, Fe²⁺, Fe³⁺), anions (e.g., CO₃²⁻, SO₄²⁻), amino acids (such as glycine), lipids, phospholipids, fatty acids and esters thereof, surfactants, triglycerides, bile acids and conjugates and salts thereof (e.g., cholic acid, deoxycholic acid, glycocholate, taurocholate, sodium cholate), and any polymers disclosed herein.

The preformed microparticles may optionally be separated from the solution and washed prior to the surface modification as disclosed herein, or be surface-modified without separation or washing. Separation means include, without limitation, centrifugation, dialysis, sedimentation (creaming), phase separation, chromatography, electrophoresis, precipitation, extraction, affinity binding, filtration, and diafiltration. For active agents with relatively low aqueous solubility, the washing medium may be aqueous, optionally containing one or more solubility reducing agents (SRAs) and/or excipients as disclosed herein. Preferred SRAs are capable of forming insoluble complexes with the active agents and/or carriers in the microparticles, and include, without limitation, compounds such as salts that comprise divalent or polyvalent cations (such as those disclosed herein). For active agents with relatively high aqueous solubility (such as proteinaceous compounds), the washing medium may be organic, or aqueous but containing at least one SRA or precipitating agent (such as ammonium sulfate). In one example, the washing medium is the same solution used in the phase separation reaction, such as an aqueous solution including approximately 16% (w/v) PEG and 0.7% (w/v) NaCl.

It is preferred that the washing medium has a low boiling point for easy removal by, for example, lyophilization, evaporation, or drying. The washing medium may be a supercritical fluid or a fluid near its supercritical point, used alone or in combination with a co-solvent. Supercritical fluids may be solvents for the PSEAs, but not for the preformed microparticles. Non-limiting examples of supercritical fluids include liquid CO₂, ethane, and xenon. Non-limiting examples of co-solvents for use in combination with a supercritical fluid include acetonitrile, dichloromethane, ethanol, methanol, water, and 2-propanol.

As indicated above, active agents with varying degrees of solubility in water may be employed in the microparticles described herein. While water insoluble active agents may be used, water-soluble active agents are preferred.

The active agent may be a pharmaceutical agent. Depending on its effect and/or application, the pharmaceutical agent includes, without limitation, adjuvants, adrenergic agents, adrenergic blocking agents, adrenocorticoids, adrenolytics, adrenomimetics, alkaloids, alkylating agents, allosteric inhibitors, anabolic steroids, analeptics, analgesics, anesthetics, anorexiants, antacids, anti-allergic agents, antiangiogenesis agents, anti-arrhythmic agents, anti-bacterial agents, antibiotics, antibodies, anticancer agents, anticholinergic agents, anticholinesterases, anticoagulants, anticonvulsants, antidementia agents, antidepressants, antidiabetic agents, antidiarrheals, antidotes, antiepileptics, antifolics, antifungals, antigens, antihelmintics, antihistamines, antihyperlipidemics, antihypertensive agents, anti-infective agents, anti-inflammatory agents, antimalarials, antimetabolites, antimuscarinic agents, antimycobacterial agents, antineoplastic agents, antiosteoporosis agents, antipathogen agents, antiprotozoal agents, adhesion molecules, antipyretics, antirheumatic agents, antiseptics, antithyroid agents, antiulcer agents, antiviral agents, anxiolytic sedatives, astringents, beta-adrenoceptor blocking agents, biocides, blood clotting factors, calcitonin, cardiotonics, chemotherapeutics, cholesterol lowering agents, cofactors, corticosteroids, cough suppressants, cytokines, diuretics, dopaminergics, estrogen receptor modulators, enzymes and cofactors thereof, enzyme inhibitors, growth differentiation factors, growth factors, hematological agents, hematopoietics, hemoglobin modifiers, hemostatics, hormones and hormone analogs, hypnotics, hypotensive diuretics, immunological agents, immunostimulants, immunosuppressants, inhibitors, ligands, lipid regulating agents, lymphokines, muscarinics, muscle relaxants, neural blocking agents, neurotropic agents, paclitaxel and derivative compounds, parasympathomimetics, parathyroid hormone, promoters, prostaglandins, psychotherapeutic agents, psychotropic agents, radio-pharmaceuticals, receptors, sedatives, sex hormones, sterilants, stimulants, thrombopoietics, trophic factors, sympathomimetics, thyroid agents, vaccines, vasodilators, vitamins, xanthines, as well as conjugates, complexes, precursors, and metabolites thereof. The active agent may be used individually or in combinations of two or more thereof. In one example, the active agent is a prophylactic and/or therapeutic agent that includes, but is not limited to, peptides, carbohydrates, nucleic acids, other compounds, precursors and derivatives thereof, and combinations of two or more thereof.

As discussed above, the active agent may be a cosmetic agent. Non-limiting cosmetic agents include inter-alia emollients, humectants, free radical inhibitors, anti-inflammatories, vitamins, depigmenting agents, anti-acne agents, antiseborrhoeics, keratolytics, slimming agents, skin coloring agents and sunscreen agents. Non-limiting compounds useful as cosmetic agents include linoleic acid, retinol, retinoic acid, ascorbic acid alkyl esters, polyunsaturated fatty acids, nicotinic esters, tocopherol nicotinate, unsaponifiables of rice, soybean or shea, ceramides, hydroxy acids such as glycolic acid, selenium derivatives, antioxidants, beta-carotene, gamma-orizanol and stearyl glycerate. The cosmetic agents may be commercially available and/or prepared by known techniques.

As discussed above, the active agent may be a nutritional supplement. Non-limiting nutritional supplements include proteins, carbohydrates, water-soluble vitamins (e.g., vitamin C, B-complex vitamins, and the like), fat-soluble vitamins (e.g., vitamins A, D, E, K, and the like), and herbal extracts. The nutritional supplements may be commercially available and/or prepared by known techniques.

As discussed above, the active agent may be a compound having a molecular weight of 2 kD or less. Non-limiting examples of such compounds include steroids, beta-agonists, anti-microbials, antifungals, taxanes (antimitotic and antimicrotubule agents), amino acids, aliphatic compounds, aromatic compounds, and urea compounds.

In one example, the active agent may be a therapeutic agent for prevention and/or treatment of pulmonary disorders. Non-limiting examples of such agents include steroids, beta-agonists, anti-fungals, anti-microbial compounds, bronchial dialators, anti-asthmatic agents, non-steroidal anti-inflammatory agents (NSAIDS), AAT, and agents to treat cystic fibrosis. Non-limiting examples of steroids include beclomethasone (such as beclomethasone dipropionate), fluticasone (such as fluticasone propionate), budesonide, estradiol, fludrocortisone, flucinonide, triamcinolone (such as triamcinolone acetonide), flunisolide, and salts thereof. Non-limiting examples of beta-agonists include salmeterol xinafoate, formoterol fumarate, levo-albuterol, bambuterol, tulobuterol, and salts thereof. Non-limiting examples of anti-fungal agents include itraconazole, fluconazole, amphotericin B, and salts thereof.

As discussed above, the active agent may be a diagnostic agent. Non-limiting diagnostic agents include x-ray imaging agents and contrast media. Non-limiting examples of x-ray imaging agents include ethyl 3,5-diacetamido-2,4,6-triiodobenzoate (WIN-8883, ethyl ester of diatrazoic acid); 6-ethoxy-6-oxohexyl-3,5-bis(acetamido)-2,4,6-triiodobenzoate (WIN 67722); ethyl-2-(3,5-bis(acetamido)-2,4,6-triiodobenzoyloxy)-butyrate (WIN 16318); ethyl diatrizoxyacetate (WIN 12901); ethyl 2-(3,5-bis(acetamido)-2,4,6-triiodobenzoyloxy)propionate (WIN 16923); N-ethyl 2-(3,5-bis(acetamido)-2,4,6-triiodobenzoyloxy-acetamide (WIN 65312); isopropyl 2-(3,5-bis(acetamido)-2,4,6-triiodobenzoyloxy)acetamide (WIN 12855); diethyl 2-(3,5-bis(acetamido)-2,4,6-triiodobenzoyloxymalonate (WIN 67721); ethyl 2-(3,5-bis(acetamido)-2,4,6-triiodobenzoyloxy)phenyl-acetate (WIN 67585); propanedioic acid, [[3,5-bis(acetylamino)-2,4,5-triodobenzoyl]oxy]bis(1-methyl)ester (WIN 68165); and benzoic acid, 3,5-bis(acetylamino)-2,4,6-triodo-4-(ethyl-3-ethoxy-2-butenoate)ester (WIN 68209). Preferred contrast agents desirably disintegrate relatively rapidly under physiological conditions, thus minimizing any particle associated inflammatory response. Disintegration may result from enzymatic hydrolysis, solubilization of carboxylic acids at physiological pH, or other mechanisms. Thus, poorly soluble iodinated carboxylic acids such as iodipamide, diatrizoic acid, and metrizoic acid, along with hydrolytically labile iodinated species such as WIN 67721, WIN 12901, WIN 68165, and WIN 68209 or others may be preferred.

As discussed above, the active agents may be used in a combination of two or more thereof. Non-limiting examples include a steroid and a beta-agonist, e.g., fluticasone propionate and salmeterol, budesonide and formoterol, etc.

Preformed microparticles may be substantially free of internal voids and/or cavities (such as being free of vesicles), substantially free of encapsulation, substantially free of lipids, substantially free of hydrogel or swelling, substantially non-porous, amorphous, solid, and/or spherical as those terms are defined herein. Preformed microparticles may have multiple surface channel openings, the diameter of which are generally 100 nm or less, preferably 10 nm or less, more preferably 5 nm or less, most preferably 1 nm or less. Preformed microparticles may have an overall density of 0.5 g/cm³ or greater, preferably 0.75 g/cm³ or greater, more preferably 0.85 g/cm³ or greater. The density may be generally up to about 2 g/cm³, preferably 1.75 g/cm³ or less, more preferably 1.5 g/cm³ or less.

Preformed microparticles may exhibit a high payload of the at least one active agent. Depending on the formulation and the physical/chemical nature of the compounds, there are typically at least 1000 or more, such as a few million to hundreds of millions of the active agent molecules in each of the preformed microparticles. The weight percentage of the active agent in the preformed microparticle may be any of the amounts below or greater, or any ranges there between, but less than 100%, for example: 10%, 15%, 20%, 25%, 30%, 35%, 40%, 45%, 50%, 55%, 60%, 65%, 70%, 75%, 80%, 85%, 90%, 95%, 98%, and 99%. While incorporation of a significant amount of bulking agents and/or other excipients is not required in the preformed microparticles, one or more of such compounds may be present therein. In any event, the desired integrity and/or activity are retained for a majority (50% or greater, preferably 75% or greater, more preferably 90% or greater, most preferably 95% or greater) of the active agent, if not 100%.

Surface modification of the preformed microparticles is achieved, without limitation, by forming, in a controlled manner, at least one monolayer containing at least one charged compound about the preformed microparticle. Examples of surface-modified microparticles and methods of forming the same are set forth in U.S. Patent Publication No. 2006/0260777 entitled “Surface-Modified Microparticles and Methods of Forming and Using the Same,” in the name of Rashba-Step et al., the entire contents of which is incorporated herein by reference. When two or more such monolayers are formed, each typically contains different charged compounds, and preferably each carries on its outer surface a net surface charge that is different in sign and/or value from that of the preceding one and/or the subsequent one, if present. Deposition of such monolayers one at a time allows for optimal control over various properties of the resulting microparticles, allowing one to tailor or “fine-tune” the microparticles to achieve a desired result.

Preferably, the monolayer immediately about the preformed microparticle (“formed monolayer”) contains one or more charged compounds, each independently having a net charge that is opposite in sign to the net surface charge of the core microparticle. The preformed microparticle may at least, in part, be penetrable by the charged compound in the formed monolayer. An outer surface of the formed monolayer may carry a net surface charge that is different from, preferably opposite in sign to, that of the preformed microparticle outer surface, especially when the formed monolayer is a saturated monolayer as defined herein. The charged compounds may include one or more of polyelectrolytes, charged polyaminoacids, charged polysaccharides, polyionic polymers, charged proteinaceous compounds, charged peptides, charged lipids optionally in combination with uncharged lipids, charged lipid structures, and derivatives thereof.

The surface-modified microparticle may further contain one or more additional alternating charged monolayers, such that the surface-modified microparticle has a desired release profile of the active agent. This number is not particularly limited, but may typically be between 1 to 7, such as 2, 3, 4, 5, or 6. Optionally, one or more of such charged monolayers may independently have one or more of the same or different active agents, such as an affinity molecule, especially a targeting ligand, associated covalently and/or non-covalently thereto, preferably on their respective outer surfaces. Alternatively or in combination, the core microparticle may have one or more portions, such as a center or an underlying layer (a charged monolayer, for example), containing at least one such active agent, preferably on the outer surface of the portion.

The preformed microparticle, the surface-modified microparticle, and any intermediates there between, if any, may be and/or have one or more of the following characteristics: spherical as defined herein, free of covalent crosslinking, free of hydrogel and/or swelling, and have a polydisperse or, preferably, monodisperse, size distribution. The preformed microparticle may be free of lipids and/or encapsulation.

Preferably, the surface-modified microparticle is capable of controlled release, especially sustained release, of the active agent, with a non-limiting release profile such as an initial burst and a linear release profile, and may be provided as a suspension or a dry powder in compositions or formulations for pharmaceutical, therapeutic, diagnostic, cosmetic, and/or nutritional applications. As discussed above, the controlled release may occur within a selected pH environment. In that regard, preferably the controlled release may occur within a pH range of approximately 2 to 10, and more preferably approximately 5 to 7.5, such as a physiological pH of 7 to 7.4 or endosomal pH of 5 to 6.5.

Controlled deposition of the one or more monolayers may further involve alteration of the net surface charge of the microparticle (such as the preformed microparticle onto which one or more of the monolayers have been deposited) through a controlled manipulation of one or more conditions, such as changes in temperature, pressure, pH, ionic strength and/or osmolality of the reaction medium, concentrations of components within the reaction medium, the likes thereof, as well as rates of such changes, and combinations of two or more thereof. Such controlled manipulations may desirably be applied before and up to the deposition of the one or more monolayers, or even during the monolayer formation. In one example, the net surface charge of the microparticle is capable of being positive, neutral, and negative. The net surface change is selected through, for example, a controlled change in one or more of the conditions described above, such as a controlled change in pH. In one example, the pH of the solution is selected such that the net surface charge of the microparticle is negative, and the difference between the pH of the solution and the surface-neutral point of the microparticle is less than 0.3, alternatively equal to or greater than 0.3, preferably 0.5 or greater, more preferably 0.8 or greater, most preferably 1 or greater.

In one exemplary method of providing surface-modified microparticles in accordance with the present disclosure, a suspension of a plurality of preformed microparticles used as three-dimensional substrates for the deposition is first provided. Non-limiting methods of forming the preformed microparticle include those disclosed herein and any other methods known to those of skill in the art. One such method involves providing a solution containing the active agent and the phase-separation enhancing agent, inducing a liquid-solid phase separation through, for example, controlled cooling, and forming the preformed microparticle. In one example, any one, two, or more, or all of the compounds used to form the preformed microparticles may preferably be distributed homogeneously throughout each preformed microparticle (e.g., being present at similar concentrations in the center, on the surface, and anywhere else therein). It will be understood that methods of surface modification as disclosed herein may be incorporated in whole or in part into the underlying methods of fabricating the preformed microparticles or made to be a continuation thereof. Between the pre-formation of the unmodified microparticle and the surface modification, the preformed microparticle may be separated from the liquid phase and, optionally, washed, preferably in the presence of the phase-separation enhancing agent. For example, the washing medium may be the same solution used during phase separation, containing the phase-separation enhancing agent. Alternatively, the preformed microparticle is not separated from the liquid phase or washed. In any event, the suspension or a re-suspension of the preformed microparticle is combined and mixed with a solution that includes at least one suitable charged compound.

As described above, the preformed microparticles may have a weight percent (wt. %) loading of the active agent of 40% or more, preferably 60% or more, or 80% or more, or 90% or more, or 95% or more, and less than 100%, typically 98% or less. The preformed microparticles may further have, or are capable of being induced (such as from a neutral state) to have, a net surface electric charge. In one example, the net surface charge is contributed primarily or essentially by the active agent and/or the carrier, if any, present in the preformed microparticles; the compound(s) may preferably be homogeneously distributed therein. Alternatively, the active agent is compartmentalized in one or more portions of the preformed microparticle, such as a center or an underlying layer (a charged monolayer, for example), preferably distributed substantially homogeneously within the portion or primarily on an outer surface thereof. The preformed microparticles may be exposed to (such as mixed with) at least one charged compound having or capable of having a net electrical charge that is, preferably, opposite in sign to the net surface charge of the preformed microparticle, thereby forming the formed monolayer of the charged compound about the preformed microparticle. The formed monolayer or the surface modified microparticle has a net surface electric charge that may be the same in sign as that of the preformed microparticle, zero or, preferably, opposite in sign to that of the preformed microparticle. In other words, if the outer surface of the preformed microparticle has a negative net surface charge (such as determined by zeta-potential measurements), then the formed monolayer may preferably have on its outer surface a positive net surface charge. Alternatively, if the preformed microparticle has a positive net surface charge, then the formed monolayer may preferably have a negative net surface charge. Deposition of the monolayer can take place in an aqueous medium (e.g., water, buffer, or aqueous solution containing some water miscible organic solvent of the type previously described, or one that may be present in the manufacture of the preformed microparticle).

To prepare the surface-modified microparticle, a non-limiting method includes preforming or otherwise providing an unmodified microparticle, exposing it to one or more charged compounds, which may be provided in a solution into which the microparticle may be immersed, and forming the monolayer. The solution may contain one or more of water, a buffer, and a water-miscible organic solvent, and one or more solubility reducing agents (e.g., alcohols, carbohydrates, non-ionic aqueous-miscible polymers, and/or inorganic ionic compounds containing monovalent or polyvalent cations), with a concentration in weight-to-volume percentage of 5% to 50%, preferably 10% to 30%. A non-limiting example of the solution contains about 16% (w/v) polyethylene glycol and 0.7% (w/v) NaCl. The pH of the solution, typically in a range of 4 to 10, may be adjusted to be same as or close to the surface-neutral point of the core microparticle (such as with a difference of 0 to less than 0.3), or away from that (such as with a difference of 0.3 pH units or greater). The charged compound may be present in the solution at a concentration of 0.05 mg/mL to 10 mg/mL. The preformed microparticle and the charged compound are co-incubated in the solution, preferably at a temperature of 2° C. to 5° C. or up to ambient temperature over a period of 1 second to 10 hours. The formation of the monolayer may be carried out in a controlled manner. The resulting surface-modified microparticle or an intermediate thereof may be separated from the solution with optional washing. The washing medium may be the same as the solution described above. The procedure may be repeated using alternating charged compounds to form the alternating charged monolayers, if desired.

As indicated above, the reaction system can include one or more solubility reducing and/or viscosity increasing agents (SRA/VIA), as well as one or more PSEAs. Suitable SRA/VIAs and PSEAs include, without limitation, those known to one skilled in the art and those disclosed herein, such as alcohols (e.g., ethanol, glycerol), carbohydrates (such as sucrose), non-ionic aqueous-miscible polymers (e.g., PEG, PVP, block copolymers of polyoxyethylene and polyoxypropylene (poloxamers), hetastarch, dextran, etc.), and inorganic ionizable compounds containing polyvalent (e.g., divalent, trivalent) cations (e.g., metal and organic cations such as those disclosed herein), such as ZnCl₂.

Thus, in one example, deposition of the formed monolayer may take place in a solution that includes buffered saline (that is, 0.7% NaCl buffer) and 8% or more by weight or volume of a SRA/VIA such as PEG, preferably 12% or more, more preferably 15% or more; typically 30% or less, preferably 25% or less, more preferably 20% or less, most preferably about 16% or more. The amount of SRA/VIA required in the solution will depend, in part, on the stability of the active agent, as well as the dissolution profile of the monolayer(s). Certain charged compounds (such as the polycations gelatin B and chitosan) may work in solutions containing 16% or less SRA/VIA.

The pH of the solution at which the net surface charge of the microparticle is zero is referred herein as the surface-neutral point of the microparticle in the particular solution. In certain examples, the pH of the solution may be adjusted to be at or near the surface-neutral point of the microparticle in the solution, with a difference there between of less than 0.3 (pH units), preferably 0.25 or less, more preferably 0.2 or less. In other examples, the pH of the solution may preferably be adjusted away from the surface-neutral point of the microparticle in the solution, with a difference there between of 0.3 (pH units) or greater, preferably 0.5 or greater, more preferably 0.8 or greater, most preferably 1 or greater. It has been observed that in certain examples, adjusting the solution pH away from the surface-neutral point of the microparticle can affect dissolution kinetics of the active agent therein. Incubation of the microparticles in the solution can be performed at or, preferably, below ambient temperature, but preferably above the freezing temperature of the solution, to minimize disintegration of the microparticles. Incubation temperature may even be lower than the freezing temperature of the solution when one or more FPDAs disclosed herein are used. For example, the incubation temperature may be between 0° C. and 15° C., preferably between 1° C. and 110° C., more preferably between 2° C. and 5° C., most preferably less than 5° C. In general, the concentration of the charged compound in the solution for each monolayer fabrication may be equal to, less than, and/or greater than one of the following, or in a range between any two thereof: 0.05 mg/mL, 0.1 mg/mL, 0.5 mg/mL, 1 mg/mL, 10 mg/mL, 5 mg/mL, 3 mg/mL. When the preformed microparticle is co-incubated with the charged compound in the solution, a weight ratio of the preformed microparticle to the charged compound may be 1:1 or greater, preferably 2:1 to 10:1, more preferably 2.5:1 to 7:1

Incubation time may be adjusted to achieve the desired charge modification (such as neutralization or charge reversal), monolayer coverage, and/or monolayer thickness. Depending on the particular reaction (such as ingredients and/or conditions), the incubation time may be equal to, shorter than, and/or longer than one of the following, or in a range between any two thereof: 10 hours, 5 hours, 3 hours, 10 minutes, 30 minutes, 100 minutes, 75 minutes, 60 minutes, 15 minutes, 5 minutes, 1 minute, 30 seconds, 10 seconds, 5 seconds, 1 second. Each monolayer may have a thickness that is equal to, less than, and/or greater than one of the following, or in a range between any two thereof: 100 nm, 50 nm, 20 nm, 5 nm, 1 nm, 0.5 nm, 0.1 nm, 2 nm, 10 nm. A typical monolayer of the present disclosure is less than 100 nm in thickness, preferably less than 10 nm.

Without wishing to be bound by any particular theory, it is believed that a factor in controlling release of the active agent from the microparticles may be the type and/or degree of interaction and/or association (e.g., non-covalent association, ionic complexation) that occurs at or near the outer surface of the preformed microparticle (such as the interface with the formed monolayer), which may involve the active agent, the charged compound, and/or other components, if any. In some cases, a strong interaction or association at this interface slows down, delays, and/or otherwise hinders dissolution of the active agent, and is believed to stabilize the surface-modified microparticle and facilitate fabrication of additional alternating charged monolayers, if desired. In addition, as described in greater detail below, the interaction can be further affected by the subsequent formation of additional alternating charged monolayers.

Thus, co-incubation of the preformed microparticles and a charged compound, preferably in a solution, results in intermediate microparticles with a single monolayer of the charged compound formed on and associated with at least the outer surface of the preformed microparticle. Following incubation, the suspension of the intermediate microparticles may then be separated from the solution through centrifugation, filtration, diafiltration, and/or other separation methods. The intermediate microparticles are optionally washed with a washing solution (preferably an aqueous medium, such as the SRA-containing buffer described above). The temperature during the incubation and the optional washing are optimized based on the solubilities of the active agent and the charged compound.

If further surface modification is desired or required, after the optional washing, the intermediate microparticle may be further exposed to (such as mixed with) a different charged compound, preferably in a solution, to form a subsequent monolayer of the different charged compound about and associated with the previously formed monolayer of the intermediate microparticle. The different charged compound preferably has a net electric charge opposite in sign to that of the previously deposited charged compound. The subsequent monolayer may be formed immediately about the formed monolayer. The latter intermediate microparticle thus formed may have a net surface electric charge that is the same in sign as that of the former intermediate microparticle, neutral or, preferably, opposite in sign to that of the former intermediate microparticle. The monolayer formation procedure may be repeated as in the previous cycle, to form further microparticles that have additional and, preferably but not necessarily, adjacent alternating charged monolayers, each associated with the preceding monolayer. The total number of the monolayers to be formed may be selected or predetermined such that controlled release of the active agent with a desired release profile is achievable in the surface-modified microparticle. As set forth above, this number can be an integer of 1, 2, 3, 4, 5, 6, 7, or greater, preferably 100 or less, more preferably 20 or less, and most preferably 10 or less.

In another example, one or more of the charged compounds forming the monolayers may be active agent(s) identical to or different from the one in the preformed microparticle. For example, one or more of the odd numbered (e.g., first, third) monolayers may independently be formed of the same or different charged active agent(s), having net electric charge(s) opposite in sign to the net surface charge of the preformed microparticle. Alternatively or in combination, one or more of the even numbered (e.g., second, forth) monolayers may independently be formed of the same or different charged active agent(s), having net electric charge(s) same in sign as the net surface charge of the preformed microparticle. The latter charged compound may be an active agent that is the same as or different from the one in the preformed microparticle, and the former charged compound may be an otherwise inert charged compound or a charged active agent different from the one in the preformed microparticle.

In another example, one or more active agents, charged and/or uncharged, may be incorporated into one or more of the monolayers through covalent and/or non-covalent associations. Such monolayer-bound active agent(s) may be the same as that of the preformed microparticle, or different there from. Such a construction may allow controlled release (e.g., extended release, sustained release) of the monolayer-bound active agent(s). Alternatively or in combination, one or more of such monolayer-bound active agent(s) may be affinity molecules, such as targeting ligands, which may selectively bring the underlying microparticle to a predetermined region to achieve targeted delivery of the active agent within the core microparticle.

In a further example, the surface-modified microparticles described above having one or more monolayers of charged compounds, preferably in a suspension, may undergo one or more physical and/or chemical treatments to further modify one or more characteristics of the surface-modified microparticles, such as, but not limited to, the release profile of the active agent therein. The treatments may be carried out immediately after the formation of the surface-modified microparticles and prior to any optional washing, or immediately following the optional washings. The treatment may involve manipulation of one or more parameters of the reaction mixture, such as, without limitation, temperature, pH, and/or pressure. Typically, the one or more parameters may be adjusted (such as increased or decreased) from an initial value to a second value and held for a period of time, and then adjusted (such as decreased or increased) to a third value or returned or allowed to return to the initial value and held for another period of time.

The thermal treatment, for example, may involve a heating stage and a cooling stage. Prior to the additional treatment, the suspension may be kept at a relatively low temperature below ambient temperature to at least minimize dissolution of the microparticles therein, preferably the temperature at which the surface-modified microparticles are formed, more preferably 2° C. to 10° C., such as 4° C. During the heating stage, the suspension may be heated to a temperature and incubated at this elevated temperature for an incubation period of 1 minute to 5 hours, preferably 15 minutes to 1 hour, such as 30 minutes. The elevated temperature may be higher than the relatively low temperature at which the suspension was kept prior to the additional treatment, and lower than a degradation temperature of the surface-modified microparticles in the suspension, preferably between 5° C. and 40° C., more preferably between 10° C. and 30° C. The heating stage may optionally be immediately followed with a cooling stage, during which the suspension may be chilled at a temperature, rapidly or gradually in a controlled manner and optionally incubated at this depressed temperature for an incubation period of 1 minute to 5 hours, preferably 15 minutes to 1 hour, such as 30 minutes. In one example, chilling is achieved by washing with a chilled washing solution. Alternatively, the suspension may be allowed to return to or close to its original temperature or to a selected temperature below the temperature to which the suspension was heated. The depressed temperature may be lower than the elevated temperature, and higher than a freezing temperature of the suspension, preferably at or below ambient temperature, optionally equal to or different from the relatively low temperature at which the suspension was kept prior to the additional treatment, more preferably 15° C. or lower, most preferably 10° C. or lower, such as 4° C. The resulting mixture may further undergo optional washings as described herein to yield additionally treated, surface-modified, microparticles.

Surface-modified microparticles suitable for the additional treatment described above include those formed from amorphous, solid, and homogenous preformed microparticles having 40% to less than 100%, or more typically 80% or greater, by weight, of an active agent as described herein. Non-limiting examples of suitable suspensions include microparticles (such as insulin microspheres) in a buffer, such as a PEG buffer containing 16% PEG, 0.7% NaCl, 67 mM Na acetate, and having a pH in the range of 5 to 8 (e.g., 5.7, 5.9, 6.5, 7.0). The microparticles may have a concentration in the buffer of 33-0.01 mg/ml to 50 mg/ml, preferably 0.1 mg/ml to 10 mg/ml, such as 1 mg/ml. A charged compound or a mixture of two or more thereof, such as protamine sulfate, poly-L-lysine, and/or poly-L-arginine, may be mixed into the suspension to provide a concentration of 0.01 mg/ml to 10 mg/ml, preferably 0.1 mg/ml to 1 mg/ml, such as 0.3 mg/ml. The mixture may be incubated at the relatively low temperature, such as 4° C., and under agitation for an incubation period of 10 seconds to 5 hours, such as 1 hour, to ensure the formation of a monolayer of the charged compound on the outer surface of each of the preformed microparticles. Then the suspension may be subjected to the thermal treatment as described above. Optional washings may be carried out on the suspension prior to the additional treatment.

The additional treatments may be carried out immediately after the formation of any one or more of the monolayers as disclosed herein. In one example, the additional treatment may be carried out immediately after the formation of a single monolayer on the preformed microparticles, the monolayer being formed of positively charged compounds or negatively charged compounds. When optionally one or more additional monolayers are formed on the first monolayer, the additional treatment may or may not be carried out immediately following the formation of such additional monolayers. In another example, two or more monolayers may be formed sequentially on the core microparticles, and the additional treatment may be carried out only immediately after a single predetermined monolayer (such as the last monolayer, the first monolayer, or any other monolayer there between) is formed. In a further example, two or more monolayers may be formed sequentially on the core microparticles, and the additional treatment may be carried out immediately after the formation of each and every monolayer having one or more predetermined characteristics, such as containing positively charged or negatively charged compounds, or containing a particular compound (e.g., active agent, affinity molecule, derivative) or moiety (e.g., functional group, label), or being a particular monolayer from the core (e.g., first, second, third, fourth, fifth). In a further example, the additional treatment may be carried out immediately after the formation of each monolayer of a predetermined set, which may be all of the monolayers or a subset thereof.

The surface-modified microparticles following the additional treatment may display modifications in net surface charge (zeta potential) and/or release profile of the active agent therein. With certain charged compounds (such as PLL and PLA, but not protamine sulfate), a change (such as an increase) in the surface charge of the surface-modified microparticles may be observed. When subjected to the in vitro release protocol as disclosed herein, the additionally treated, surface-modified microparticles are capable of displaying a reduction in the 1-hour percentage of cumulative release (% CR₁ₕ) of the active agent therein, as compared to the surface-modified microparticles without the additional treatment. Inasmuch as it is believed that the initial burst of the active agent release typically occurs within the first hour, the example demonstrates that the initial burst of the active agent release may be significantly reduced by the additional treatment. The same additionally treated, surface-modified microparticles may be capable of continued, preferably sustained, release beyond 1 hour, preferably beyond 24 hours, more preferably beyond 48 hours, most preferably beyond 7 days, having a 24-hour percentage of cumulative release (% CR₂₄ₕ) that is greater than % CR₁ₕ. As a result of the additional treatment, the surface-modified microparticles of the present disclosure, when subjected to in vitro release in a release buffer (10 mM Tris, 0.05% Brij 35, 0.9% NaCl, pH 7.4, free of divalent cation) at 37° C., may be capable of displaying a % CR₁ₕ of 50% or less and/or a ratio of % CR₂₄ₕ to % CR₁ₕ of greater than 1:1. The % CR₁ₕ may preferably be 40% or less, more preferably 30% or less, further preferably 20% or less, most preferably 10% or less. The ratio of % CR₂₄ₕ to % CR₁ₕ may preferably be 1.05:1 or greater, more preferably 1.1:1 or greater, but not more than 10:1, preferably 5:1 or less, more preferably 2:1 or less, most preferably 1.5:1 or less.

Without being bound to any particular theory, it is believed that the additional treatment following the monolayer formation as disclosed herein allows the charged compound in the monolayer and the molecules (e.g., the active agent, the optional carrier molecules in the preformed microparticle, the charged compound in the preceding monolayer) that comprises the outer surface of the substrate (e.g., the preformed microparticle, the preceding monolayer) to rearrange and form an association that is much stronger than the electrostatic interaction between the monolayer and the outer surface of the substrate prior to the additional treatment. It is believed that through the additional treatment a modified shell is formed on the outer surface of the surface-modified microparticle, the modified shell containing a homogenous mixture of the charged compound and the molecules that form the outer surface of the substrate.

Deposition of additional alternating charged monolayers of charged compounds beyond the formed monolayer may further affect, among other things, the release profile of the active agent in the preformed microparticle. As previously described, depending on the attractive forces at the interface between the preformed microparticle and the formed monolayer, strong association between the two may be observed. This may result in retarding the quantity and/or rate of release of the active agent. The release profile may be further modified by forming one or more additional alternating charged monolayers about the formed monolayer. Without being restricted to any particular theory, it is believed that addition of a second oppositely charged monolayer may ease the association between the formed monolayer and the preformed microparticle, thereby enhancing the release of the active agent. Subsequent application of the alternating charged monolayers, arranged consecutively with optional interleaving layers of active agents, if desired, can allow fine-tuning of active agent release from the surface-modified microparticles, as shown in some of the examples disclosed herein.

Suitable charged compounds that may be used in accordance with the present invention may be charged compounds capable of associating with any substrate, preferably by, but not limited to non-covalent association and, more preferably, electrostatic interaction. Thus, suitable charged compounds include positively charged, negatively charged, or zwitterionic, and include, but are not limited to, polyelectrolytes, charged polyaminoacids, polysaccharides, polyionic polymers, ionomers, charged peptides, charged proteinaceous compounds, charged lipids optionally in combination with uncharged lipids, charged lipid structures such as liposomes, precursors and derivatives thereof, and combinations of two or more thereof. Non-limiting examples include negatively charged polyelectrolytes such as polystyrene sulfonate (PSS) and polyacrylic acid (PAA), negatively charged polyaminoacids such as polyaspartic acid, polyglutamic acid, and alginic acid, negatively charged polysaccharides such as chondroitin sulfate and dextran sulfate, positively charged polyelectrolytes such as polyallyl amine hydrochloride (PAH) and poly(diallyldimethyl ammonium chloride (PDDA), positively charged polyaminoacids such as poly(L-lysine) hydrochloride, polyornithine hydrochloride, and polyarginine hydrochloride, and positively charged polysaccharides such as chitosan and chitosan sulfate. Also useful as charged compounds in the present invention are, without limitation, biocompatible polyionic polymers (e.g., ionomers, polycationic polymers such as polycationic polyurethanes, polyethers, polyesters, polyamides; polyanionic polymers such as polyanionic polyurethanes, polyethers, polyesters, polyamides), charged proteins (e.g., protamine, protamine sulfate, xanthan gum, human serum albumin, zein, ubiquitins, and gelatins A & B), and charged lipids (e.g., phosphatidyl choline, phosphatidyl serine). Also included are derivatives (e.g., glycosylated, hyperglycosylated, PEGylated, FITC-labeled, salts thereof), conjugates, and complexes of the charged compounds disclosed herein. More specifically, suitable positively charged lipids (that is, polycationic lipids), negatively charged lipids (that is, polyanionic lipids), and zwitterionic lipids include 1,2-distearoyl-sn-glycero-3-phosphocholine (DSPC), 1,2-dioleoyl-sn-glycero-3-phosphoethanolamine-N-(carboxyfluorescein) (FITC-EA), 1,2-distearoyl-sn-glycero-3-[phospho-rac-(1-glycerol)] (sodium salt) (DSPG), 1,2-dipalmitoyl-sn-glycero-3-phosphate (monosodium salt) (DPPA), and 1,2-dioleoyl-3-dimethylammonium-propane.

Furthermore, lipid structures (such as liposomes) can be used in alternate deposition with charged compounds. Uncharged (such as non-ionic) lipids may be used in combination with electrically charged lipids to form one or more of the monolayers, and the molar ratio there between can be optimized to achieve minimum permeability of the active agent through the monolayer.

The surface-modified microparticle disclosed herein, typically containing a core microparticle and one or more monolayers, preferably has a release profile of the active agent that is different from that of the core microparticle. Non-limiting examples of the differences in release profile include a reduction in the initial burst, an extension of release time, a display of linear/constant release over a time period, and/or a reduction in rate of release over a prolonged time period. The surface-modified microparticles may be present, preferably in a functionally (e.g., therapeutically, pharmaceutically, diagnostically) effective amount, as a suspension or dry powder in a liquid or solid composition or formulation, in the presence or absence of one or more of preservatives, isotonicity agents, pharmaceutically acceptable carriers, and stabilizing agents. Such compositions and formulations may be administered in an effective amount to a subject for prevention or treatment of a condition or state, or as a nutritional supplement, or for the purpose of physical enhancement or psychological well-being. Such compositions and formulations may be incorporated into a diagnostic method, tool, or kit for in vitro and/or in vivo detection of a substance, condition, or disorder being present or absent, or a disposition for such a condition or disorder. For example, the substance, upon contact, may form an association (e.g., conjugate, complex) with the surface-modified microparticle or a portion thereof (such as the core microparticle), which is capable of providing one or more signals for detection. The one or more signals may be one or more moieties labeled on one or more portions of the association (e.g., the substance, the microparticles), or may be elicited upon the formation of the association (e.g., emission of light, discharge of another substance). Additionally, the surface-modified microparticles may be incorporated into a nutritional and/or dietary supplement or a food composition, or used as a food additive, for prevention and/or treatment of a condition or disorder in a subject.

The surface-modified microparticles or the core microparticles disclosed herein, alone or in a mixture thereof (with a weight ratio there between of values including 1:99, 10:90, 20:80, 25:75, 30:70, 40:60, 50:50, 60:40, 70:30, 75:25, 80:20, 90:10, 99:1, or within a range between any two of such values), can further be encapsulated within matrices of one or more matrix-forming materials to form microencapsules. The microencapsulation can be accomplished by using any one or more processes known in the art, including solution precipitation, anti-solvent precipitation, spray drying, spray freezing (into vapor or liquid), evaporative precipitation into aqueous medium, and emulsification/solvent extraction processes, such as those disclosed in U.S. Patent Publication Nos. 2006/0121121, 2005/0170005, 2005/0142205, 2004/0110871, 2004/0018229, 2003/0059402, 2003/0041602, and 2002/0081334, the entireties of which are incorporated herein by express reference thereto. Non-limiting examples of emulsion systems useful to encapsulate the various microparticles disclosed herein include single-emulsion systems (such as solid-in-water-in-oil, solid-in-oil-in-water, solid-in-water-in-water, and solid-in-oil-in-oil) and multiple-emulsion systems (such as double emulsion, like S/W/O/W and S/O/W/O, triple emulsion, and even more complex emulsion systems). Non-limiting techniques for solvent extraction useful in combination with the various emulsion systems include lyophilization, vacuum drying, freeze-drying, liquid-phase drying, spray drying such as cold spray drying, cryogenic drying, spray-freeze drying, supercritical drying such as supercritical fluid drying, and fluidized bed drying, used alone or in combinations of two or more thereof. In one example, microencapsulation of the surface-modified microparticles and/or the core microparticles is accomplished by an emulsification/solvent extraction process as described below. The matrix can impart additional sustained release properties to the active agent resulting in release rates that persist for minutes, hours, days, weeks, months, or even longer, according to the desired therapeutic applications. The microencapsules can also produce delayed release formulations using the surface-modified microparticles and/or the core microparticles.

The examples described herein demonstrate that, unexpectedly, microencapsulation of the surface-modified microparticles provides a synergistic effect in reducing the initial burst of in vitro release (Ibᵥₜ) of the active agent. Ib_{vt-c}, Ibᵥₜ₋ₛ, Ib_{vt-ec}, and Ibᵥₜ₋ₑₛ are the cumulative releases in percentage of the active agent from a core microparticle, a surface-modified microparticle containing the same core microparticle, a microencapsule containing the same core microparticle, and a microencapsule containing the same surface-modified microparticle, respectively, over an initial period of, for example, 24 hrs or less, such as 12 hrs or less, or 6 hrs or less, or 1 hr. It has been found herein that typically Ib_{vt-ec} is much less than Ibᵥₜ₋ₛ and one typically could expect from this finding that Ibᵥₜ₋ₑₛ would be about the same as the lesser of Ibᵥₜ₋ₛ and Ib_{vt-ec}. Therefore, it is surprising that the data shown herein demonstrating a combination of microencapsulation and surface modification as disclosed herein provides a Ibᵥₜ₋ₑₛ that is much less than Ivₜ₋ₛ. In fact, Ibᵥₜ₋ₑₛ is typically less than or equal to the multiple of Ibᵥₜ₋ₛ and Ib_{vt-ec}. In one example, one or more of 24-hr Ibᵥₜ₋ₑₛ, 12-hr Ibᵥₜ₋ₑₛ, 6-hr Ibᵥₜ₋ₑₛ, and 1-hr Ibᵥₜ₋ₑₛ of the microencapsule is 10% or less, such as 5% or less, 3% or less, or 1% or less. Furthermore, it is unexpected that the reduction in burst found in the microencapsules containing one or more surface-modified microparticles also has a high loading (i.e., high content by weight) of the at least one active agent present in the surface-modified microparticles. The active agent loading is at least 5%, such as greater than, equal to, or less than 10%, 15%, 20%, 25%, 30%, 35%, 40%, 45%, 50%, 60%, or in a range between any of such values.

A further unexpected result of the combination of surface-modification followed by encapsulation is that high levels of bioavailability and/or bioactivity of the active agent are retained. The core microparticle formation and the surface-modification process are carried out in the absence of organic solvents and/or extreme physiochemical stress. With the protection of the one or more monolayers on the surface-modified microparticles, the active agent therein is prevented from being subjected to organic solvents and/or physiochemical stresses during the encapsulation process, which can denature or otherwise modify the active agent and render it inactive.

Particles of the present disclosure such as microencapsules include those containing at least one discontinuous phase dispersed in a continuous phase, such as solid dispersions, and those containing two or more physiochemically distinct materials, such as composites. The at least one discontinuous phase is surface-modified, such as in the manner disclosed herein, but not limited thereto. The at least one discontinuous phase can be a composite having two or more different materials. Typically, at least one of the discontinuous phase and the continuous phase is amorphous; preferably, both are amorphous. In one example, the two or more different materials in the microencapsule include at least one compound that is capable of forming a conforming or non-conforming intermolecular self-assembly and/or has a net non-zero charge, present in an amount of W₁; at least one macromolecule and/or active agent present in an amount of W₂; and at least one matrix-forming material in an amount of W₃, where W₁, W₂, and W₃ are all weights of the same unit or percentages by weight of the microencapsules (i.e., W₁+W₂+W₃≦100%). Typically, W₁<W₂, W₁<W₃, and W₂≦W₃, while in certain cases W₃<W₂. W₁ can be 50% of W₂ or less, such as 25% or less, 10% or less, 5% or less, 1% or less, or in a range between any two of such values. W₁ can be 50% of W₃ or less, such as 10% or less, 5% or less, 1% or less, or in a range between any two of such values. W₂ can be 50% or less of W₃, such as 30% or less, 15% or less, 10% or less, 6% or less, 4% or less, 2% or less, 1% or less, or in a range between any two of such values. In one example, the charged compound of the present disclosure is not a surfactant. In another example, the macromolecule and/or active agent is not a surfactant.

The at least one macromolecule and/or active agent is present in a discontinuous phase in the microencapsule, such as in the form of a plurality of microparticles (like the surface-modified microparticles and/or the core microparticles as described herein) that are preferably discrete and dispersed (i.e., non-agglomerated and non-aggregated) as well as spherical. The discontinuous phase is typically solid, and can be amorphous, composite, or otherwise non-crystalline, and may contain nanocrystalline and/or amorphous structures (such as those of the macromolecule and/or the active agent) therein. The at least one macromolecule is an active agent, and/or the discontinuous phase further contains one or more active agents (may or may not be macromolecules themselves). The active agents may be nanocrystalline and/or amorphous, and are releasable from the microencapsule into a medium. The at least one matrix-forming material is present in a continuous phase in the microencapsule, such as a matrix that is preferably solid and amorphous, and the matrix may or may not be crosslinked and/or a hydrogel. The continuous phase (such as the matrix and the matrix-forming material therein) encapsulates the discontinuous phase (such as the plurality of microparticles and the macromolecules and/or active agents therein).

The at least one charged and/or self-assemblable compound is disposed in one or more of the following manners: at an outermost boundary of the discontinuous phase (being part of the discontinuous phase, such as on the outer surface of the microparticles); about the discontinuous phase (such as about the plurality of microparticles and/or about the at least one macromolecule and/or active agent); in between the discontinuous phase (such as the plurality of microparticles and/or the at least one macromolecule and/or active agent) and the continuous phase (such as the matrix or the at least one matrix-forming material); and/or in association with the plurality of microparticles and/or a portion (some but not all) of the at least one macromolecule and/or active agent. As a result, the at least one charged and/or self-assemblable compound partially or fully divides the discontinuous phase from the continuous phase by partially or fully covering the discontinuous phase; alternatively or in combination, the at least one charged and/or self-assemblable compound is part of the discontinuous phase without being homogenously distributed therein. In one example, the at least one charged and/or self-assemblable compound is present in one or more self-assemblies (preferably conforming ones) and/or one or more monolayers (preferably saturated ones) disposed about (thereby partially or fully covering) the core microparticles and/or the at least one macromolecule and/or active agent. In another example, one or more of the at least one charged and/or self-assemblable compound, the self-assembly, and the monolayer is exposed to (e.g., being in contact with, being dispersed in, being encapsulated by) one or more of the at least one matrix-forming material and the matrix. When the at least one macromolecule and/or active agent is one or more proteinaceous compounds and/or nucleic acids, the at least one charged compound can have a positive net charge.

The microencapsule has an active agent content by weight (Cₑ) of 50% or less, such as being greater than, equal to, or less than a value of 40%, 30%, 25%, 20%, 18%, 15%, 14%, 13%, 12%, 10%, 8%, 5%, 3%, 2%, 1%, 0.5%, or in a range between any two of such values. The discontinuous phase (such as the plurality of microparticles with and/or without one or more of the charged and/or self-assemblable compounds, the self-assemblies, and the monolayers) has a content of the same active agent by weight (C_{d}) of 20% or more but less than 100%, such as being greater than, equal to, or less than a value of 30%, 40%, 50%, 60%, 70%, 75%, 80%, 85%, 90%, 95%, 98%, 99%, or in a range between any two of such values. Alternatively, C_{d} is 15% or less, such as being greater than, equal to, or less than a value of 13%, 10%, 8%, 5%, 3%, 2%, 1%, 0.5%, or in a range between any two of such values. The continuous phase has a content of the same active agent by weight (C_{c}) that is different from Cₑ and/or C_{d}; typically, C_{c}≦Cₑ and/or C_{c}≦C_{d}. In one example, the continuous phase is substantially free of the active agent (e.g., C_{c}≦1%, C_{c}=0%) and/or the macromolecule. Typically, C_{c} is different from C_{d}, such as Cₑ<C_{d}, with a ratio of Cₑ:C_{d} of 1:2 or less, such as being greater than, equal to, or less than a value of 1:3, 1:4, 1:5, 1:7, 1:9, 1:10, 1:20, 1:30, 1:40, 1:50, 1:60, 1:70, 1:80, 1:90, 1:100, or in a range between any two of such values.

The microencapsule (ES) containing the surface-modified discontinuous phase (containing the core microparticles) dispersed in the matrix releases the at least one active agent with an initial burst of IBₑₛ. The same surface-modified discontinuous phase (such as the surface-modified microparticles containing the same core microparticles) without encapsulation releases the same active agent with an initial burst of IBₛ. The same core microparticles, without encapsulation or surface-modification, releases the same active agent with an initial burst of IB_{c}. Another microencapsule (EC) containing the same core microparticles (without surface-modification) dispersed in the same matrix releases the same active agent with an initial burst of IB_{ec}. Typically, IBₑₛ<IB_{c}, IBₛ<IB_{c}, and IB_{ec}<IB_{c}, provided that all the initial bursts are above quantitation level. In one example, IBₑₛ≦IB_{ec} and/or IBₑₛ≦IBₛ. In another example, IBₑₛ≦IBₛ×IB_{ec}. The microencapsule (ES) displays an extended release of the active agent following the initial burst substantially the same as that of the microencapsule (EC).

When mixtures of the surface-modified microparticles and the unmodified core microparticles (with a weight ratio there between of Wₛ:W_{c}) are encapsulated in the same matrix, their respective initial bursts are within a range between IBₑₛ and IB_{ec}, while their respective extended release phases are substantially the same as that of the microencapsules (ES) and (EC). As such, the release profile of the active agent from the microencapsule can be fine-tuned by adjusting, for example, the ratio Wₛ:W_{c}, without being limited thereto. The ratio Wₛ:W_{c} is in a range of 99:1 to 1:99, such as greater than, equal to, or less than a value of 95:5, 90:10, 85:15, 80:20, 75:25, 70:30, 67:33 (equivalent to 2:1), 60:40, 55:45, 50:50, 45:55, 40:60, 33:67 (equivalent to 1:2), 30:70, 25:75, 20:80, 15:85, 10:90, 5:95, or in a range between any two of such values. In one example, the ratio Wₛ:W_{c} is correlated (such as in a substantially linear manner) with the percentages of cumulative release of the active agent from the microencapsules over a period of greater than 24 hours, such as 7 days or longer. The releases of the active agent from the different formulations are measured in the same manner when comparisons are made, which may be in vitro release into a predetermined medium (such as a buffer), or in vivo release in a certain type of subjects (such as rats). Initial bursts are measured over the same period of time, typically over a time period of 24 hours or less, such as 6 hours or 1 hour.

In an exemplary emulsification/solvent extraction process, an emulsion is obtained by mixing two immiscible or partially miscible phases (i.e., a continuous phase and a discontinuous/dispersed phase). In one example, the continuous phase is a water phase, an aqueous phase, or an aqueous-miscible phase, while the discontinuous/dispersed phase is aqueous-immiscible or partially aqueous miscible (e.g., an oil phase, a water-immiscible organic phase, a partially water-miscible organic phase), and the two phases upon mixing form an oil-in-water (O/W) emulsion. In another example, the discontinuous/dispersed phase is a solid-in-oil (S/O) phase (i.e., a dispersion, such as a suspension) containing particles (such as the surface-modified microparticles and/or the core microparticles disclosed herein) dispersed in one or more water-immiscible and/or partially water-miscible organic solvents that are non-solvent(s) to the dispersed particles. In a further example, a reverse emulsion, such as a water-in-oil (W/O) emulsion, forms from an aqueous-immiscible or partially aqueous miscible continuous phase and a discontinuous/dispersed phase that is aqueous or aqueous-miscible. Regardless of whether the discontinuous/dispersed phase is a suspension of S/W or S/O, a ratio by weights of the discontinuous/dispersed phase to the continuous phase can range from about 1:99 to about 99:1, such as being greater than, equal to, or less than values chosen from 2:98, 5:95, 10:90, 15:85, 20:80, 25:75, 30:70, 35:65, 40:60, 45:55, 50:50, 60:40, 70:30, 80:20, and 90:10, or in a range between any two of such values. Other non-limiting emulsions suitable for encapsulating the particles of the present disclosure include solid-in-water-in-water (S/W/W) emulsions, solid-in-oil-in-oil emulsions (S/O/O), and those having more than two phases, such as solid-in-oil-in-water-in-oil (S/O/W/O) emulsions, and solid-in-water-in-oil-in-water (W/O/W) emulsions.

In one example, a discontinuous/dispersed phase is provided, containing the surface-modified microparticles and/or the core microparticles of the present disclosure suspended or otherwise dispersed in an organic solution (such as methylene chloride) that contains at least one matrix-forming materials (such as PLGA). This solid-in-oil (S/O) phase can be formed with the use of a homogenizer, an impeller, a spinning oil film (as described in Leach, et al., AAPS Pharm Sci Tech. 2005; 6(4): E605-E617), or any other effective mixing/shearing means. Water or an aqueous solution (e.g., salt solution, surfactant solution, buffer, binary or ternary mixtures containing water) is provided as the aqueous continuous phase. By emulsifying (vigorously mixing/shearing, e.g., by homogenization, spinning, or sonication) the S/O phase with the aqueous phase, a solid-in-oil-in-water (S/O/W) emulsion system is formed, containing emulsified droplets of embryonic microencapsulated particles.

The aqueous continuous phase can be pre-saturated with the same organic solvent contained in the S/O phase prior to emulsification to minimize rapid extraction of the organic solvent from the emulsified droplets. The emulsification process can be performed at any temperature at which the mixture can maintain its liquid properties. Stability of the emulsion is a function of the concentration of the surface active compound in the organic phase or in the aqueous phase, or in the emulsion if the surface active compound is added to the emulsion after the emulsification process. Non-limiting factors affecting the average particle size and the size distribution of the resulting solid microencapsules include viscosity of the continuous phase, viscosity of the discontinuous phase, shear forces during emulsification, type and concentration of the surface active compound, and the weight ratio between the phases.

Following emulsification, the emulsion is transferred into a hardening medium. The hardening medium extracts the solvent in the discontinuous/dispersed phase from the embryonic microencapsulated particles, resulting in the formation of solid microencapsules having the surface-modified microparticles and/or the core microparticles dispersed in a solid, amorphous polymeric matrix of the matrix-forming material. In an O/W or S/O/W emulsion system, the hardening medium can be an aqueous medium, which may optionally contain one or more surface active compounds, thickening agents, and/or other excipients. To moderate the extraction time and/or the extraction rate, heating (e.g., through exposure to conduction or convection heat, radiation such as IR, microwave, or radiowave, or other heating means or energy source) and/or pressure reduction can be applied to the mixture including the hardening medium and the emulsified phases. The extraction rate of solvent in the discontinuous phase from the embryonic microencapsulated particles is a factor in the degree of porosity in the final solid microencapsules, since rapid removal, e.g., by evaporation (boiling effect), of the discontinuous phase tends to result in destruction of the continuity of the matrix.

The microencapsules can have any one or more physical characteristics of the microparticles disclosed herein (including the preformed microparticles and the surface-modified microparticles), such as being substantially spherical, having a particle size of from about 0.6 μm to about 300 μm, from about 0.8 μm to about 60 μm, from about 1 μm to about 10 μm, from about 10 μm to about 30 μm, or from about 1 μm to about 5 μm, and/or having a particle size distribution as disclosed herein.

In one example, the emulsification process is performed in a continuous fashion instead of a batch process. In another example, the hardened polymeric matrices, encapsulating the surface-modified microparticles and/or the core microparticles of the active agent, are further washed and collected using centrifugation and/or filtration (including diafiltration). Residual liquids can be removed, if desirable, using techniques such as lyophilization, evaporation, freeze-drying, spray-drying, freeze-spray-drying, and other liquid removal or drying means known to one of ordinary skill in the art.

The matrix-forming material refers to materials capable of forming the structural entity of the amorphous matrix individually or in combination. Biodegradable and biocompatible matrix-forming materials are suitable for injectable applications, among others. Non-limiting examples of suitable matrix-forming materials include the family of poly-lactide/poly-glycolide copolymers (PLGA's), polyethylene glycol conjugated PLGA's (PLGA-PEG's), block copolymers of polyethylene oxide and poly-lactide/poly-glycolide (such as PLGA-PEO-PLGA), block copolymers of poloxamers and poly-lactide/poly-glycolide (such as PLGA-poloxamer-PLGA), homopolymers and copolymers of polylactic acids, and triglycerides. Homopolymers and copolymers of poly-lactide and/or poly-glycolide can be terminated with, without limitation, ester, acid, hydroxyl, amine, methyl, methoxy, thiol, maleimide, and/or sulfhydryl groups. In the examples of homopolymers and copolymers of poly-lactide and/or poly-glycolide (e.g., PLGA or PLGA-PEG), a ratio of poly-lactide to poly-glycolide can be from 100:0 to 0:100, such as from about 90:10 to about 15:85, or about 50:50. In general, the higher the ratio of the poly-glycolide to the polylactide in the polymer, the more hydrophilic the microencapsules, resulting in faster hydration and faster degradation. Molecular weight of the polymers is not particularly limited, and can range from 1 kD to 1,000 kD, such as 5 kD, 10 kD, 20 kD, 24 kD, 30 kD, 35 kD, 50 kD, 80 kD, 100 kD, 200 kD, 500 kD, or a range between any such values. In general, for microencapsulation matrices formed from copolymers with the same ratio of poly-glycolide to poly-lactide, the higher the molecular weight of the copolymer, the slower is the release of the active agent from the matrix, and the wider the distribution of the size of the microencapsules.

The organic solvent in the organic phase (oil phase) of an O/W or S/O/W emulsion can be aqueous-immiscible or partially aqueous-immiscible. Suitable aqueous-immiscible solvents include, but are not limited to: substituted or unsubstituted, linear, branched or cyclic alkanes, alkenes, and alkynes with a carbon number of 5 or higher; aromatic hydrocarbons; completely or partially halogenated hydrocarbons; ethers; esters; certain ketones (such as fatty ketones, but not include acetone); mono-, di- or tri-glycerides; natural oils; certain alcohols (such as fatty alcohols, but not include methanol or ethanol); aldehydes; certain acids (such as fatty acids), certain amines; linear or cyclic silicones; hexamethyldisiloxane; or any combination of these solvents. Halogenated solvents include, but are not limited to: carbon tetrachloride, methylene chloride, chloroform, tetrachloroethylene, trichloroethylene, trichloroethane, hydrofluorocarbons, chlorinated benzene (mono, di, tri), and trichlorofluoromethane. Non-limiting examples of partially aqueous-miscible solvents are tetrahydrofuran (THF), propylene carbonate, benzyl alcohol, and ethyl acetate, which are of limited water miscibility and capable of spontaneous emulsion formation. Particularly suitable non-limiting solvents are methylene chloride, chloroform, diethyl ether, toluene, xylene, and ethyl acetate.

One or more surface active compounds can be present in the emulsion, for example, to increase the wetting properties of the organic phase. The surface active compounds can be added before the emulsification process, to either the continuous phase or the discontinuous phase or both (depending in part on their solubility in the different phases), or into the emulsion after it is formed. The use of surface active compounds can reduce the number of unencapsulated or partially encapsulated microparticles, thereby reducing the initial burst of the active agent during its release. Non-limiting examples of surface active compounds include ionic surfactants (e.g., cationic, anionic, zwitterionic), nonionic surfactants, and surface active biological molecules. The surface active compound should be present (e.g., in the continuous phase, the discontinuous phase, and/or the emulsion) in an amount by weight of less than, equal to, or greater than values such as 0.01%, 0.05%, 0.1%, 0.5%, 1%, 5%, 10%, 15%, 30%, or in a range between any two of such values.

Non-limiting examples of cationic surfactants include quaternary ammonium compounds, such as benzalkonium chloride, cetyltrimethylammonium bromide, lauryldimethylbenzylammonium chloride, acyl carnitine hydrochlorides, and alkyl pyridinium halides. Non-limiting examples of anionic surfactants include salts of fatty acids, potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.), and phospholipids. Non-limiting examples of phospholipids include phosphatidylcholine, phosphatidylethanolamine, phosphatidylserine, phosphatidyl inositol, phosphatidylglycerol, phosphatidic acid, lysophospholipids, egg or soybean phospholipid or a combination thereof. The phospholipid may be salted or desalted, hydrogenated or partially hydrogenated or natural, semisynthetic or synthetic.

Non-limiting examples of nonionic surfactants include: polyoxyethylene fatty alcohol ethers (Macrogol and Brij), polyoxyethylene sorbitan fatty acid esters (Polysorbates), polyoxyethylene fatty acid esters (Myrj), sorbitan esters (Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, polyvinyl alcohol, polyvinylpyrrolidone, and polysaccharides (including starch and starch derivatives such as hydroxyethylstarch (HES), methylcellulose, hydroxycellulose, hydroxy propylcellulose, hydroxy propylmethylcellulose, and noncrystalline cellulose). Poloxamers under the trade name PLURONIC® are available from Spectrum Chemical and Ruger. Among polyoxyethylene fatty acid esters is included those having short alkyl chains, such as SOLUTOL® HS 15 (polyethylene-660-hydroxystearate) available from BASF Aktiengesellschaft. Non-limiting examples of surface active biological molecules include albumin, casein, heparin, hirudin, hetastarch, and other appropriate biocompatible agents. In one example, the aqueous continuous phase contains a nonionic surfactant (such as polysaccharides, like methylcellulose) or a surface active biological molecule (such as proteins, like albumin).

Certain compounds listed as surface active compounds above may also function as excipients in the particles and/or compositions of the present disclosure. Non-limiting examples of excipients include saccharides, disaccharides (such as sucrose, trehalose), and sugar alcohols (such as mannitol). Use of channeling agents, such as polyethylene glycol (PEG), can increase water permeability of the final product, which results in modification of the release kinetics of the active agent. Incorporation of the channeling agent during encapsulation can be advantageous in terms of simplifying and/or reducing washing when the same compounds (such as PEG) is used elsewhere during the process (such as the phase-separation enhancing agent during the formation of the core microparticles). Salinity (through the use of certain salts, for example) and/or pH of the continuous phase can be varied to affect properties of the resulting microencapsules, such as matrix packing density, surface charge, wetting, porosity, viscosity, particle size distribution, as well as initial burst and release kinetics of the encapsulated active agent from the matrix. Salinity of the continuous phase can also be used to reduce miscibility of the two phases. Non-limiting examples of salts useful to adjust salinity include water-soluble phosphates, sulfates, acetates, and carbonates, Tris (Tris(hydroxymethyl)aminomethane), MES (2-[N-Morpholino]ethanesulfonic acid), and HEPES (N-[2-hydroxyethyl]piperazine-N′-[2-ethanesulfonic acid]). Salt concentration can range from 0 to 10 M, such as from 1 mM to 1 M, or from 20 to 200 mM. The pH can range from 1 to 11, such as from 2.5 to 9, or from 6 to 8.

The microencapsules disclosed herein, typically containing a plurality of the surface-modified microparticle and/or the core microparticles, preferably has a release profile of the active agent that is different from that of the surface-modified microparticle and that of the core microparticle. Non-limiting examples of the differences in release profile include a reduction in the initial burst, an extension of release time, a display of linear/constant release over a time period, and/or a reduction in rate of release over a prolonged time period. The microencapsules may be present, preferably in a functionally (e.g., therapeutically, pharmaceutically, diagnostically) effective amount, as a suspension or dry powder in a liquid or solid composition or formulation, in the presence or absence of one or more of preservatives, isotonicity agents, pharmaceutically acceptable carriers, and stabilizing agents. Such compositions and formulations may be administered in an effective amount to a subject for prevention or treatment of a condition or state, or as a nutritional supplement, or for the purpose of physical enhancement or psychological well-being Such compositions and formulations may be incorporated into a diagnostic method, tool, or kit for in vitro and/or in vivo detection of a substance, condition, or disorder being present or absent, or a disposition for such a condition or disorder. For example, the substance, upon contact, may form an association (e.g., conjugate, complex) with the microencapsules or a portion thereof (such as the core microparticle), which is capable of providing one or more signals for detection. The one or more signals may be one or more moieties labeled on one or more portions of the association (e.g., the substance, the microparticles), or may be elicited upon the formation of the association (e.g., emission of light, discharge of another substance). Additionally, the microencapsules may be incorporated into a nutritional and/or dietary supplement or a food composition, or used as a food additive, for prevention and/or treatment of a condition or disorder in a subject. In a further example, the in vivo release profile is that of the microencapsule administered through injection, such as subcutaneous, intravenous, or intramuscular injections. The microencapsules can be formulated to have smooth surface and/or being substantially spherical.

Compositions containing the microencapsules containing one or more surface-modified microparticles can be used as sustained release formulations of the active agent for various in vivo administrations (e.g., injection, inhalation, ingestion, infusion). The sustained release formulations can be therapeutically or prophylactically effective over a period of at least one day, such as one week, two weeks, one month, two months, three months, six months, or even one year. For injectable compositions, the microencapsules containing one or more surface-modified microparticles can have a maximum diameter of 200 microns or less, such as equal to or less than 125 microns, 100 microns, 90 microns, 60 microns, 30 microns, or in a range between any such values. To further enhance the injectability of the composition, a diluent may be used as a liquid carrier for the microencapsules. The diluent can contain one or more solubility-reducing agents such as those disclosed herein above.

To further modify the release kinetics of the microencapsules, the concentration of the charged compounds used to form the surface-modified microparticles encapsulated therein can be adjusted if desirable. In one example, the concentration is reduced to form an unsaturated monolayer about the core particle, thereby altering the release kinetics of the resulting surface-modified microparticles as well as that of the microencapsules formed there from.

Set forth below are examples of microencapsules fabricated in accordance with the present disclosure. All of the charged monolayers formed in the examples are believed to be saturated monolayers as described herein. Readings and measurements reported were recorded using instruments and methods described below.

### In Vitro Release (IVR)

To generate the IVR profile of the active agent (such as insulin), a 10 mL aliquot of a releasing buffer (10 mM Tris, 0.05% Brij 35, 0.9% NaCl, pH 7.4) was added into a glass vial containing 0.5 mL of the concentrated particle suspension (equivalent to 3 mg of insulin), mixed, and incubated at 37° C. At designated time intervals 400 μL of the IVR medium was transferred into a microfuge tube and centrifuged for 2 minutes at 13 k rpm. A 300 μL aliquot of the supernatant was removed and stored at −80° C. until analyzed by Bicinchoninic Acid (BCA) assay as understood by one skilled in the art. A 300-μL aliquot of fresh releasing buffer was added to the microfuge tube to reconstitute the pellet. The 400-μL suspension was transferred back to the IVR. Total active agent content of the microparticle was determined by BCA assay after complete dissolution of the microparticle in an aqueous alkaline solution containing dimethyl sulfoxide (DMSO) and a surfactant and pH neutralization.

### EXAMPLES

### Example 1

### Microencapsulation of Surface-Modified Microparticles

Uncoated insulin microspheres (INSms, dry powder) were resuspended in a coating buffer (16% PEG 3350, 0.7% NaCl, 67 mM sodium acetate, pH 7.0) to obtain a 8 mg/ml particle suspension. Poly-L-arginine (PLA, 15-70 kD MW) was used as the coating material. The coating solution contained 6 mg/ml of the polycationic polyaminoacid in the coating buffer. The coating process was initiated by mixing equal volumes of the particle suspension and the coating solution at 4° C., resulting in the final concentration of 4 mg/ml for the suspension, and 3 mg/ml with respect to the polycation in the reaction medium. The reaction medium was incubated at 4° C. for 30 min, followed by 30 min incubation at 15° C. At the end of the incubation period, the post-deposition coating solution was replaced with DI-water using diafiltration. The final suspension was frozen and lyophilized, and the resulting dry powder was used as primary microspheres in the microencapsulation.

A 10% solution of a 24 kD 50:50 PLGA (Medisorb 2.5A) was prepared in methylene chloride. Using a rotor/stator homogenizer, 50 mg of the PLA-coated insulin microspheres were suspended in 1.6 ml of the polymer solution to form the dispersed phase. The continuous phase consisted of aqueous solution of 0.1% (w/v) methylcellulose and 50 mM phosphate buffer at pH 7.0. The microencapsulation process was initiated by emulsification of the dispersed phase in the continuous phase in a continuous fashion. The produced emulsion was removed from the chamber and transferred into a hardening bath. The organic solvent was extracted over one hour under reduced pressure at −0.4 bar, and stirred conditions. The hardened microencapsules were collected by filtration and washed with water. The washed microencapsules were lyophilized to remove the excess water.

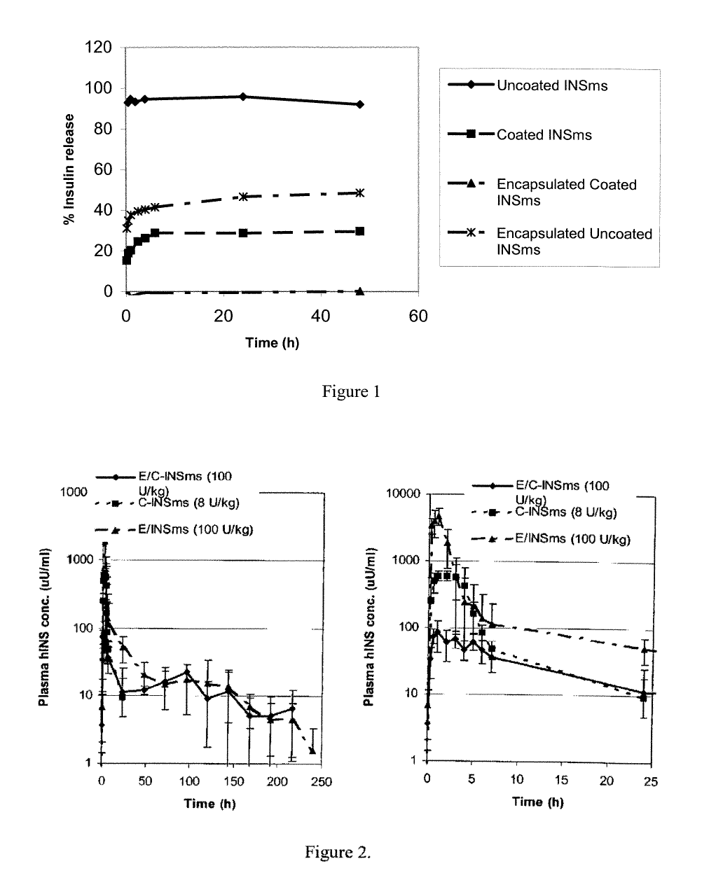

The protein content of the resultant microspheres was estimated as 14% (w/w). The in vitro release (IVR) profiles of uncoated insulin core microparticles, surface-modified microparticles formed from the same core microparticles, and their respective microencapsulated particle formulations are shown in FIG. 1 and reported in Table 1 below (BQL stands for below quantitation level). The in vivo release profiles of the same microparticles are shown in FIG. 2.

TABLE 1

| | % Cumulative Insulin Release | | | | | |
|---|---|---|---|---|---|---|
| Formulation | 1 h | 6 h | 24 h | 48 hr | 9 D | 30 D |
| Uncoated INSms | 94.7 | 94.6 | 95.8 | — | — | — |
| Coated INSms | 20.3 | 28.8 | 28.7 | 29.7 | 30.5 | 33.1 |
| Encapsulated Uncoated | 37.7 | 41.5 | 46.7 | 48.6 | 50.0 | 63.0 |
| INSms | | | | | | |
| Encapsulated Coated INSms | BQL | BQL | BQL | 0.2 | 9.8 | 49.0 |

### Example 2

### Fabrication of Microencapsules of Recombinant Human Growth Hormone (rhGH)

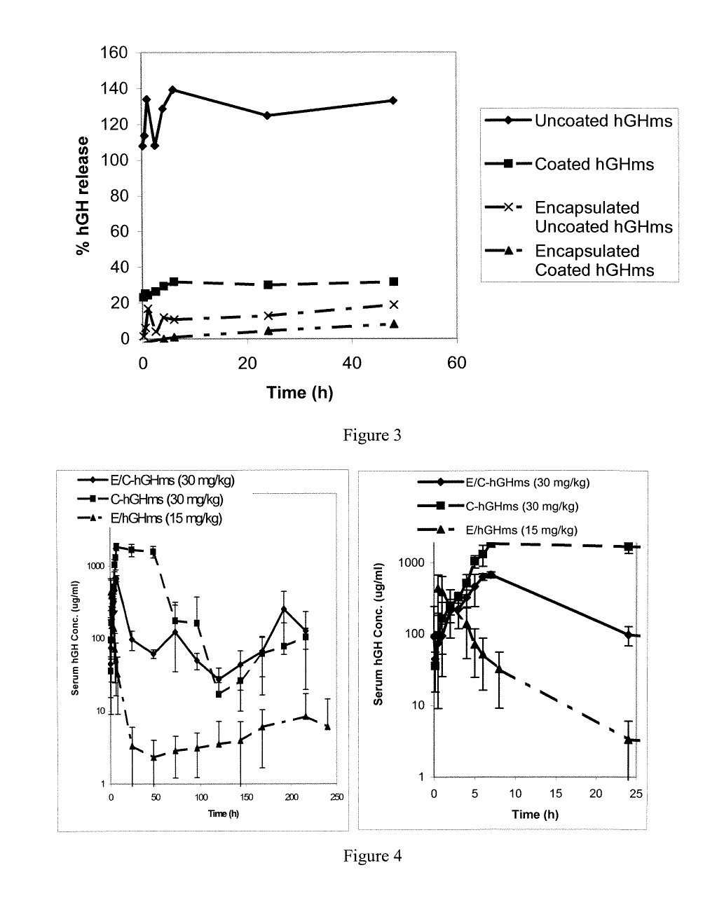

The methods and conditions as described in Example 1 were also used to produce microencapsules containing coated rhGH microspheres. The protein content of the resultant microspheres was estimated as 14.0% (w/w). The in vitro release (IVR) profiles of the uncoated rhGH core microparticles, the surface-modified microparticles formed from the same core microparticles, and their respective microencapsulated particle formulations are shown in FIG. 3 and reported in Table 2 below. The in vivo release profiles of the same microparticles are shown in FIG. 4.

TABLE 2

| | % Cumulative rhGH Release | | | | | |
|---|---|---|---|---|---|---|
| Formulation | 1 h | 6 h | 24 h | 48 hr | 9 D | 30 D |
| Uncoated hGHms | 134.1 | 139.4 | 124.5 | — | — | — |
| Coated hGHms | 24.4 | 32.0 | 30.0 | 31.7 | 32.1 | 30.2 |
| Encapsulated Uncoated | 16.8 | 10.8 | 12.9 | 18.9 | 19.4 | 36.6 |
| hGHms | | | | | | |
| Encapsulated Coated hGHms | BQL | 1.0 | 4.5 | 8.2 | 30.1 | 43.0 |

### Example 3 Microencapsulation of Mixed Microparticles

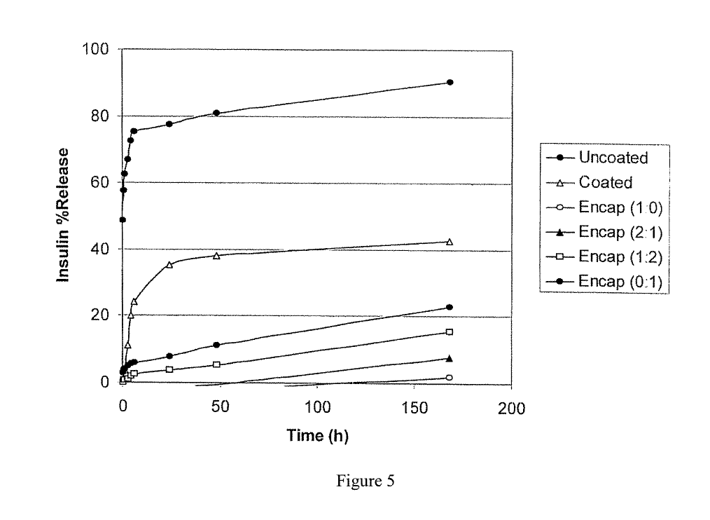

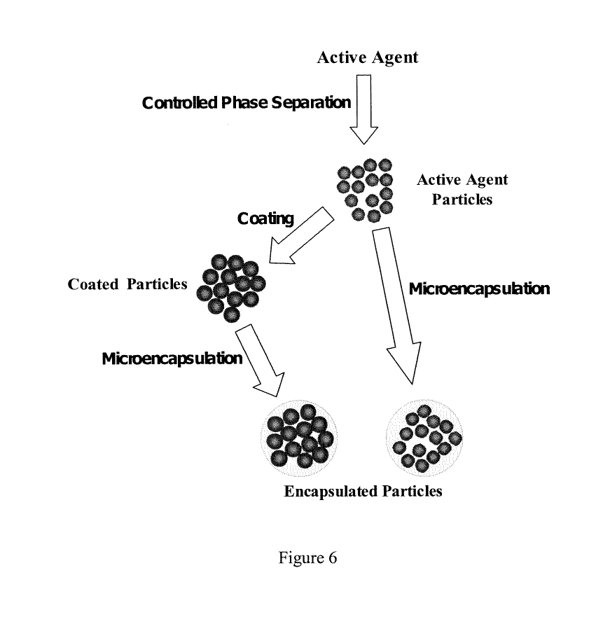

Unmodified insulin microparticles (INSms, dry powder) and surface-modified insulin microparticles were provided as in Example 1 to make 50-mg microparticle preparations, each having a weight ratio of surface-modified microparticles to unmodified microparticles being one of the following: 1:0, 1:2, 2:1, and 0:1. Each of the 50-mg microparticle preparations was encapsulated as described in Example 1. The in vitro release (IVR) profiles of the unmodified insulin microparticles, the surface-modified microparticles, and the respective microencapsules are shown in FIG. 5 and reported in Table 3 below (BQL stands for below quantitation level). The release profiles of Encapsulated (1:2) and Encapsulated (2:1) fell within the area defined by the release profiles of Encapsulated (0:1) and Encapsulated (1:0). Unexpectedly, the % cumulative release over a time greater than 24 hours, such as over 7 days, was correlated with the weight ratio of the surface-modified microparticles to the unmodified microparticles in a substantially linear fashion. By adjusting the weight ratio, a microencapsule with a desired release profile of can be prepared in accordance with the present disclosure.

TABLE 3

| | % Cumulative Insulin Release | | | | | |
|---|---|---|---|---|---|---|
| Formulation | 0.5 h | 1 h | 6 h | 24 h | 48 h | 7 D |
| Unmodified INSms | 57.7 | 62.7 | 75.1 | 77.4 | 81.0 | 90.4 |
| Surface-modified INSms | 1.3 | 3.7 | 24.2 | 35.4 | 38.0 | 42.6 |
| Encapsulated (0:1) INSms | 3.8 | 4.0 | 6.0 | 7.7 | 11.2 | 22.9 |
| Encapsulated (1:2) INSms | 0.7 | 0.7 | 2.6 | 3.8 | 5.3 | 15.6 |
| Encapsulated (2:1) INSms | BQL | BQL | BQL | BQL | BQL | 7.7 |
| Encapsulated (1:0) INSms | BQL | BQL | BQL | BQL | BQL | 1.8 |

What is claimed is:

1. A microencapsule, comprising a plurality of preformed microparticles encapsulated in an amorphous matrix, wherein at least one of the preformed microparticles comprises a core microparticle and at least one monolayer associated with the outer surface of said core microparticle, wherein the core microparticle comprises at least one active agent that is capable of being released from the microencapsule.

2. The microencapsule of claim 1, wherein the active agent comprises one or more of a bioactive agent, a pharmaceutical agent, a diagnostic agent, a nutritional supplement, and a cosmetic agent.

3. The microencapsule of claim 1, wherein the active agent comprises one or more prodrugs, affinity molecules, synthetic organic molecules, polymers, low molecular weight molecules, macromolecules, proteinaceous compounds, peptides, vitamins, steroids, steroid analogs, lipids, nucleic acids, carbohydrates, precursors thereof, and derivatives thereof.

4. The microencapsule of claim 1, wherein the preformed microparticles are substantially spherical.

5. The microencapsule of claim 1, wherein the preformed microparticles have a monodisperse size distribution.

6. The microencapsule of claim 1, wherein the preformed microparticles are amorphous and solid.

7. The microencapsule of claim 1, wherein the preformed microparticles are free of covalent crosslinking and hydrogels.

8. The microencapsule of claim 1, wherein the monolayer comprises an active agent, which may be the same or different from the active agent of the core microparticle.

9. The microencapsule of claim 8, wherein the active agent of the monolayer comprises an affinity molecule, said affinity molecule being associated with an outer surface of the monolayer.

10. The microencapsule of claim 1, wherein the preformed microparticles carry a net electric charge on an outer surface thereof, said net electric charge being positive, negative, or zero.

11. The microencapsule of claim 10, wherein said net electric charge is positive or negative.

12. The microencapsule of claim 11, wherein the monolayer comprises at least one charged compound, said charged compound having a net electric charge that is opposite in sign to the net electric charge of the preformed microparticles.

13. The microencapsule of claim 12, wherein the charged compound is selected from the group consisting of polyelectrolytes, charged polyaminoacids, polysaccharides, polyionic polymers, ionomers, charged peptides, charged proteinaceous compounds, charged lipids, charged lipid structures, precursors and derivatives thereof, and combinations thereof.

14. The microencapsule of claim 12, wherein at least one of the preformed microparticles comprises a second monolayer, said second monolayer comprising a second charged compound, said second charged compound having a net electric charge that is opposite in sign to the net electric charge of the first charged compound.

15. The microencapsule of claim 1, wherein the monolayer has a thickness of less than 100 nm.

16. The microencapsule of claim 1, wherein the amorphous matrix comprises a repeat unit selected from glycolic acid, lactic acid, triglycerides, and combinations thereof.

17. The microencapsule of claim 1, wherein the core microparticle comprises at least 80 weight percent of the active agent.

18. The microencapsule of claim 1, wherein the active agent is distributed homogeneously throughout the core microparticle.

19. A microencapsule, comprising a plurality of preformed microparticles encapsulated in an amorphous matrix, wherein at least one of the preformed microparticles comprises a core microparticle and at least one monolayer separating the core microparticle from the amorphous matrix, and the core microparticle comprises at least one active agent that is capable of being released from the microencapsule.

20. A method for preparing microencapsules comprising:
providing amorphous and solid preformed microparticles including at least one active agent and having an outer surface carrying a net electric charge;
exposing at least the outer surface of the preformed microparticles to at least one charged compound, said charged compound having a net electric charge that is opposite in sign to the net electric charge of the preformed microparticle outer surface, thereby forming surface-modified microparticles having a monolayer of the charged compound associated with the preformed microparticle outer surface; and
encapsulating one or more of the surface-modified microparticles to form microencapsules.

21. The method of claim 20, wherein the exposing step is accomplished by contacting the performed microparticles with a solution comprising the charged compound.

22. The method of claim 21, wherein the solution is selected such that the net surface charge of the preformed microparticle is negative, and the difference between the pH of the solution and the surface-neutral point of the preformed microparticle is greater than or equal to 0.3

23. The method of claim 21, wherein the concentration of the charged compound in the solution is between 0.01 mg/mL and 10 mg/mL.

24. The method of claim 20, wherein the microencapsulation is accomplished by one or more of solution precipitation, anti-solvent precipitation, spray drying, spray freezing, evaporative precipitation into aqueous medium, and emulsification/solvent extraction processes.

25. A method for preparing microencapsules comprising:
forming a solid and amorphous microparticle containing an active agent,
forming a monolayer including at least one charged compound on the formed microparticle, thereby preparing a surface-modified microparticle, and
encapsulating one or more of the surface-modified microparticles, optionally in combination with one or more of the solid and amorphous microparticles, to form microencapsules.
